(19) United States
(12) Patent Application Publication
(10) Pub. No.: US 2026/0015632 A1
(43) Pub. Date: Jan. 15, 2026

(54) CULTURE MEDIUM COMBINATION FOR INDUCING DIFFERENTIATION OF PLURIPOTENT STEM CELLS INTO CD34+ HEMATOPOIETIC STEM/PROGENITOR CELLS

(71) Applicant: SHENZHEN CELL INSPIRE BIOTECHNOLOGY CO., LTD., Shenzhen (CN)

(72) Inventors: Yubo YAN, Shenzhen (CN); Lixiang JIANG, Shenzhen (CN); Zhibin ZHU, Shenzhen (CN); Jian FU, Shenzhen (CN); Mingjun WEI, Shenzhen (CN); Meng CHU, Shenzhen (CN); Kerui ZHANG, Shenzhen (CN); Jiayin YANG, Shenzhen (CN)

(73) Assignee: SHENZHEN CELL INSPIRE BIOTECHNOLOGY CO., LTD., Shenzhen (CN)

(21) Appl. No.: 19/329,948

(22) Filed: Sep. 16, 2025

Related U.S. Application Data
(63) Continuation of application No. PCT/CN2024/093133, filed on May 14, 2024.

(30) Foreign Application Priority Data
Mar. 17, 2023 (CN) 202310292114.7

(51) Int. Cl. C12N 15/90; C07K 14/725; C07K 16/28; C12N 5/0783

(52) U.S. Cl. CPC C12N 15/907; C07K 14/7051; C07K 16/2878; C12N 5/0646; C12N 2501/125; C12N 2501/2303; C12N 2501/2307; C12N 2501/2315; C12N 2501/26; C12N 2506/45

(57) ABSTRACT

Provided are a method for inducing differentiation of pluripotent stem cells into CD34⁺ hematopoietic stem/progenitor cells and a culture medium composition thereof. Provided are a method for inducing differentiation of pluripotent stem cells into CD34⁺ hematopoietic stem/progenitor cells or NK cells and a culture medium composition thereof. The NK cells obtained using the provided culture method have high purity and good in vitro expansion effect; the yield of iNK cells is high, and a single iPSC can differentiate into approximately 2,000 NK cells; and the obtained iNK cells highly express CD16 (over 70%). As a result, the problem of low CD16 expression in iNK cells in the prior art, which requires genetic modification methods to solve, has been addressed.

### CROSS-REFERENCE TO RELATED APPLICATIONS

This application is a continuation of International Patent Application No. PCT/CN2024/093113 filed on May 14, 2024 (“PCT Application”), which claims priority to and benefits of Chinese patent application No. 202310292114.7, filed with the China National Intellectual Property Administration on Mar. 17, 2023 (“Chinese Priority Application”). The entire contents of each of the PCT Application and the Chinese Priority Application are incorporated herein by reference.

### STATEMENT REGARDING SEQUENCE LISTING

A Sequence Listing associated with this application is being filed concurrently herewith in ASCII format and is hereby incorporated by reference into the present specification. The text file containing the Sequence listing is titled “POI250931PUS-Sequence Listing”, was created on Sep. 2, 2025, and is 14,687 bytes in size.

### FIELD

The present disclosure relates to the technical field of cell engineering, and more particularly, to a method for inducing differentiation of pluripotent stem cells into CD34⁺ hematopoietic stem/progenitor cells and a culture medium composition thereof.

### BACKGROUND

Pluripotent stem cells (PSCs) possess the capacity for self-renewal and multi-lineage differentiation potential, and are capable of differentiating into almost all cell types, including hematopoietic stem/progenitor cells (HSPCs) and mature hematopoietic cells. HSPCs can further differentiate into a variety of blood cells both in vitro and in vivo, such as myeloid (My), erythroid (Er), and megakaryocytes (Mk), as well as more critical immune cells such as NK cells and T cells, thus exhibiting the potential to treat diseases. In recent years, a variety of immune cells, such as NK cells, cytotoxic T cells, Treg cells, and macrophages, have been combined with chimeric antigen receptors (CARs) to produce engineered immune cells such as CAR-NK, CAR-T, CAR-Treg, and CAR-macrophage for the treatment of tumors, infectious diseases, and autoimmune diseases. Therefore, pluripotent stem cells can be combined with CAR and directed differentiation technology to obtain HSPCs, which can then be further differentiated into engineered immune cells such as CAR-NK, CAR-T, CAR-Treg, and CAR-macrophage.

In addition, HSPC transplantation and hematopoietic cell infusion have also successfully cured some patients, particularly those with hematologic malignancies. Due to donor shortages and restricted cell numbers, HSPCs induced from pluripotent stem cells can also serve as an alternative source for hematopoietic stem cell transplantation.

However, existing methods for inducing differentiation of pluripotent stem cells into HSPCs and NK cells still face several limitations: the proportion of CD34⁺ cell populations obtained is relatively low; induction under non-3D conditions is not suitable for industrial-scale production; trophoblast cells such as mouse embryonic fibroblasts (MEFs) are still employed, rendering the methods not suitable for clinical application; and the differentiation efficiency and yield of HSPCs derived from pluripotent stem cells remain suboptimal.

Therefore, there remains a need for improved culture medium components and methods for inducing differentiation of pluripotent stem cells into HSPCs and NK cells.

### SUMMARY

The present disclosure aims to solve, at least to a certain extent, one of the technical problems in the related art. To this end, one object of the present disclosure is to provide a method for inducing differentiation of pluripotent stem cells into CD34⁺ hematopoietic stem/progenitor cells or NK cells and a culture medium composition thereof. The culture method provided by the present disclosure yields NK cells with high purity and robust in vitro expansion capability, produces high-yield induced NK (iNK) cells, with approximately 2,000 NK cells differentiated from a single iPSC, and obtains iNK cells with high CD16 expression (greater than 70%), thereby overcoming the limitation of low CD16 expression in conventional iNK cells, which typically requires genetic modification to resolve.

To this end, in a first aspect, the present disclosure provides a culture medium composition for inducing differentiation of pluripotent stem cells into CD34⁺ hematopoietic stem/progenitor cells. According to some embodiments of the present disclosure, the culture medium composition includes a first differentiation medium added in a first differentiation stage; a second differentiation medium added in a second differentiation stage; and a third differentiation medium added in a third differentiation stage.
(1) The first differentiation medium includes a first basal medium and BMP4 and a GSK-3β inhibitor;
(2) The second differentiation medium includes a second basal medium and optionally UM171, and does not include IL-3;
(3) The third differentiation medium includes a third basal medium, VEGF, bFGF, SCF, FLT3L, and TPO, and does not include IL-3.

The existing culture conditions for inducing differentiation of pluripotent stem cells into CD34⁺ hematopoietic stem/progenitor cells are complex. For example, the differentiation process requires low-oxygen culture conditions or additional sorting and enrichment steps to obtain high-purity HSCs, or involves 2D culture steps that are not suitable for closed culture systems. Furthermore, the differentiation efficiency and yield of HSPCs differentiated from pluripotent stem cells are relatively low. The inventors have found that the addition of BMP4 or a GSK-3β inhibitor, or a combination thereof, to the first differentiation medium significantly accelerates the HSPC differentiation process and enhances CD34 expression. Preferably, orbital shaker-based spheroid formation or AggreWell-based spheroid formation can be further used to yield high-purity CD34⁺ cells. Moreover, UM171 achieves optimal CD34⁺ differentiation efficiency and HSPC yield only when it is added to the second differentiation medium.

According to some embodiments of the present disclosure, the pluripotent stem cells are iPSCs or commercially available embryonic stem cells.

According to some embodiments of the present disclosure, the first basal medium includes at least one selected from mTeSR™1, TeSR™2, TeSR™-AOF, Essential 8™ Culture Media, NutriStem® hESC XF, or StemFit® Feeder-Free Stem Cell Culture Media.

According to some embodiments of the present disclosure, the first basal medium is mTeSR™1 or TeSR™-AOF medium.

According to some embodiments of the present disclosure, the second basal medium is StemPro™-34 SFM complete medium.

According to some embodiments of the present disclosure, the third basal medium is StemPro™-34 SFM complete medium.

According to some embodiments of the present disclosure, the first differentiation medium further includes a Rock inhibitor.

According to some embodiments of the present disclosure, the Rock inhibitor includes at least one selected from Y27632 or HB-100.

According to some embodiments of the present disclosure, the second differentiation medium further includes VEGF, bFGF, and BMP4.

According to some embodiments of the present disclosure, the second differentiation medium further includes an induction enhancer.

According to some embodiments of the present disclosure, the induction enhancer includes at least one selected from a GSK-3β inhibitor or SR1.

According to some embodiments of the present disclosure, the GSK-3β inhibitor includes CHIR99021, NP031112, AT7519, TWS119, SB216763, CHIR-98014, AZD1080, SB415286, LY2090314, (E/Z)-GSK-3β inhibitor 1, KY19382, Alsterpaullone, BIO-acetoxime, IM-12, 1-Azakenpaullone, or Indirubin.

According to some embodiments of the present disclosure, the second differentiation medium further includes ITS-X, β-mercaptoethanol, ascorbic acid, and GlutaMAX.

According to some embodiments of the present disclosure, the third differentiation medium further includes ITS-X, β-mercaptoethanol, ascorbic acid, and GlutaMAX.

According to some embodiments of the present disclosure, BMP4 is used at a concentration ranging from 5 ng/mL to 100 ng/m.

According to some embodiments of the present disclosure, the GSK-3β inhibitor is CHIR99021, and CHIR99021 is used at a concentration ranging from 1 μM to 20 μM.

According to some embodiments of the present disclosure, the Rock inhibitor is Y27632, and Y27632 is used at a concentration ranging from 1 μM to 20 μM.

According to some embodiments of the present disclosure, VEGF is used at a concentration ranging from 5 ng/mL to 100 ng/mL.

According to some embodiments of the present disclosure, bFGF is used at a concentration ranging from 5 ng/mL to 100 ng/mL.

According to some embodiments of the present disclosure, SCF is used at a concentration ranging from 5 ng/mL to 100 ng/mL.

According to some embodiments of the present disclosure, TPO is used at a concentration ranging from 1 ng/mL to 100 ng/mL.

According to some embodiments of the present disclosure, FLT3L is used at a concentration ranging from 1 ng/mL to 200 ng/mL, preferably from 1 ng/mL to 50 ng/mL.

According to some embodiments of the present disclosure, UM171 is used at a concentration ranging from 0 nM to 1 μM.

According to some embodiments of the present disclosure, the induction enhancer is SR1, and SR1 is used at a concentration ranging from 0 M to 2 μM.

In a second aspect, the present disclosure provides a culture medium composition for inducing differentiation of pluripotent stem cells into NK cells. According to some embodiments of the present disclosure, the culture medium composition includes a fourth differentiation medium and the first differentiation medium, the second differentiation medium, and the third differentiation medium in the culture medium composition according to the first aspect.

The fourth differentiation medium includes a fourth basal medium, IL-7, IL-15, FLT3L, SCF, and optionally IL-3.

The second differentiation medium further includes UM171.

Optionally, the second differentiation medium or the third differentiation medium further includes an SPHK2 inhibitor.

According to some embodiments of the present disclosure, the pluripotent stem cells are iPSCs or commercially available embryonic stem cells.

According to some embodiments of the present disclosure, the fourth basal medium is DMEM/F12.

According to some embodiments of the present disclosure, the SPHK2 inhibitor is ABC294640.

According to some embodiments of the present disclosure, ABC294640 is used at a concentration ranging from 1 μM to 50 μM.

According to some embodiments of the present disclosure, IL-3 is used at a concentration ranging from 0 ng/mL to 10 ng/mL.

According to some embodiments of the present disclosure, IL-7 is used at a concentration ranging from 0.1 ng/mL to 30 ng/mL.

According to some embodiments of the present disclosure, IL-15 is used at a concentration ranging from 1 ng/mL to 10 ng/mL.

According to some embodiments of the present disclosure, FLT3L is used at a concentration ranging from 1 ng/mL to 200 ng/mL, preferably from 1 ng/mL to 50 ng/mL.

According to some embodiments of the present disclosure, SCF is used at a concentration ranging from 5 ng/mL to 100 ng/mL.

According to some embodiments of the present disclosure, the fourth differentiation medium further includes human serum albumin and at least one of serum substitute, human AB serum, or FBS.

According to some embodiments of the present disclosure, the fourth basal medium further includes at least one of NAD⁺, HLA-C, SB203580, or IL-2.

According to some embodiments of the present disclosure, NAD⁺ is used at a concentration ranging from 1 μM to 500 μM.

According to some embodiments of the present disclosure, HLA-C is used at a concentration ranging from 0.05 ng/mL to 1 ng/mL.

According to some embodiments of the present disclosure, SB203580 is used at a concentration ranging from 1 μM to 50 μM.

According to some embodiments of the present disclosure, IL-2 is used at a concentration ranging from 100 IU to 5000 IU.

In a third aspect, the present disclosure provides use of the culture medium composition for inducing differentiation of pluripotent stem cells into CD34⁺ hematopoietic stem/progenitor cells according to the first aspect, or the culture medium composition for inducing differentiation of pluripotent stem cells into NK cells according to the second aspect in the preparation of NK cells.

In a fourth aspect, the present disclosure provides a method for preparing CD34⁺ hematopoietic stem/progenitor cells. According to some embodiments of the present disclosure, the method includes:
A. obtaining pluripotent stem cells in a single-cell state;
B. culturing the pluripotent stem cells in the single-cell state under a first culture condition, to obtain a first cell spheroid;
C. culturing the first cell spheroid under a second culture condition, to obtain a second cell spheroid;
D. culturing the second cell spheroid under a third culture condition, to obtain the CD34⁺ hematopoietic stem/progenitor cells.

The first culture condition includes inducing differentiation of the pluripotent stem cells using a first differentiation medium.

The second culture condition includes inducing differentiation of the first cell spheroid using a second differentiation medium.

The third culture condition includes inducing differentiation of the second cell spheroid using a third differentiation medium.

The first differentiation medium, the second differentiation medium, and the third differentiation medium are the first differentiation medium, the second differentiation medium, and the third differentiation medium in the culture medium composition for inducing differentiation of pluripotent stem cells into CD34⁺ hematopoietic stem/progenitor cells according to the first aspect.

The pluripotent stem cells are iPSCs or commercially available embryonic stem cells.

The present disclosure provides a method for differentiating pluripotent stem cells to obtain CD34⁺ cells (HSPCs). The method uses a 3D continuous culture approach, where the CD34⁺ cells concurrently express CD43 and CD44. This method is initiated by digesting pluripotent stem cells into single cells and transferring multiple pluripotent stem cells into a first differentiation medium to form a first spheroid. The first spheroid is then sequentially cultured in a second differentiation medium and a third differentiation medium, to form a second spheroid and a third spheroid. The second spheroid and the third spheroid can be cultured in a closed system. The above steps take 6 days to 14 days in total, and HSPCs are derived from the suspension cells dropped from the third spheroid. Further culturing of the HSPCs together with the third spheroid yields NK (iNK) cells. This method is suitable for culture under conventional CO₂ concentration conditions without requiring hypoxic culture conditions, and can obtain CD34⁺CD43⁺ and CD34⁺CD44⁺ cell populations with a purity of over 90% without intermediate sorting and enrichment steps. In addition, the differentiation process is free of trophoblast cells, making it suitable for the preparation of clinically used cells.

In a fifth aspect, the present disclosure provides a CD34⁺ hematopoietic stem/progenitor cell, which is obtained by the method according to the fourth aspect.

In a sixth aspect, the present disclosure provides a method for preparing NK cells. According to some embodiments of the present disclosure, the method includes:
a. obtaining CD34⁺ hematopoietic stem/progenitor cells using the method according to the fourth aspect;
b. culturing the CD34⁺ hematopoietic stem/progenitor cells under a fourth culture condition, to obtain the NK cells.

The fourth culture condition includes inducing differentiation of the CD34⁺ hematopoietic stem/progenitor cells using a fourth differentiation medium.

The fourth differentiation medium is the fourth differentiation medium in the above-mentioned culture medium composition for inducing differentiation of pluripotent stem cells into NK cells.

The second differentiation medium in the method according to the fourth aspect further includes UM171.

Optionally, the second or the third differentiation medium further includes an SPHK2 inhibitor.

Existing differentiation processes of iNK cells rely on trophoblast cells, which are unsuitable for clinical application. In addition, iNK cells exhibit a low level of CD16 expression (typically 20% to 30%) (Zhu, Huang et al. “Pluripotent stem cell-derived NK cells with high-affinity noncleavable CD16a mediate improved antitumor activity.” Blood vol. 135,6 (2020): 399-410), which limits the antibody-dependent cellular cytotoxicity (ADCC) of iNK cells. The present disclosure aims to establish a method for efficiently obtaining HSPCs, which can be further differentiated into NK cells. 1) The culture conditions are simple (normoxic culture, no additional sorting or enrichment steps, and performed entirely under 3D culture conditions); 2) The culture medium is serum-free, and no trophoblast cells are required during the differentiation process of iNK cells, which is suitable for clinical applications; 3) HSPCs exhibit high purity and yield (CD34⁺ cells with a purity of up to 99% can be obtained); 4) CD16 expression is significantly increased, reaching over 70%; and 5) iNK cells exhibit high yield, with up to a 2000-fold expansion relative to iPSCs at the end of the differentiation process. Moreover, the inventors have also found that the addition of UM171 to the second differentiation medium or the addition of an SPHK2 inhibitor to the second or third differentiation medium can significantly increase the yield of subsequent iNK cells. The addition of nicotinamide adenine dinucleotide (NAD⁺), or HLA-C, or SB203580, or IL-2 to the fourth differentiation medium further increases the yield of iNK cells without affecting the proportions of CD56⁺ or CD45⁺ cells.

According to some embodiments of the present disclosure, UM171 is used at a concentration ranging from 1 nM to 1 μM.

According to some embodiments of the present disclosure, UM171 is used at a concentration of 35 nM.

According to some embodiments of the present disclosure, the SPHK2 inhibitor includes ABC294640.

According to some embodiments of the present disclosure, ABC294640 is used at a concentration ranging from 1 μM to 50 M.

In a seventh aspect, the present disclosure provides an NK cell. According to some embodiments of the present disclosure, the NK cell is obtained by the method according to the sixth aspect.

In an eighth aspect, the present disclosure provides use of the culture medium composition for inducing differentiation of pluripotent stem cells into CD34⁺ hematopoietic stem/progenitor cells according to the first aspect, the culture medium composition for inducing differentiation of pluripotent stem cells into NK cells according to the second aspect, the CD34⁺ hematopoietic stem/progenitor cells prepared by the method for preparing CD34⁺ hematopoietic stem/progenitor cells according to the fourth aspect, the CD34⁺ hematopoietic stem/progenitor cell according to the fifth aspect, the NK cell prepared by the method for preparing NK cells according to the sixth aspect, or the NK cell according to the seventh aspect in the preparation of a CAR-NK cell.

In a ninth aspect, the present disclosure provides a CAR-NK cell. According to some embodiments of the present disclosure, the CAR-NK cell is obtained by: modifying or genetically editing pluripotent stem cells, to obtain CAR-modified pluripotent stem cells, and inducing differentiation of the CAR-modified pluripotent stem cells using the method for preparing NK cells according to the sixth aspect, to obtain the CAR-NK cell.

According to some embodiments of the present disclosure, the pluripotent stem cells are iPSCs or commercially available embryonic stem cells.

In a tenth aspect, the present disclosure provides a cell population. According to some embodiments of the present disclosure, the cell population includes the NK cell according to the seventh aspect and/or the CAR-NK cell according to the ninth aspect.

In an eleventh aspect, the present disclosure provides a medicament for preventing and/or treating a tumor. According to some embodiments of the present disclosure, the medicament includes: the NK cell according to the seventh aspect and/or the CAR-NK cell according to the ninth aspect and/or the cell population according to the tenth aspect.

The technical solution of the present disclosure has the following beneficial effects:
1) The culture conditions are simple and can be cultured under normoxic conditions, without the need for hypoxic culture;
2) CD34⁺ cells with an average purity of approximately 94% purity can be obtained;
3) CD34⁺ cells with an average of more than a 70-fold expansion relative to the initial pluripotent stem cells can be obtained;
4) NK cells obtained through further differentiation have high purity and good in vitro expansion capability;
(5) The differentiation process is performed entirely under 3D culture conditions, suitable for closed-system culture;
6) The yield of iNK cells is high, and a single iPSC can be differentiated into approximately 2,000 NK cells;
7) The iNK cells obtained in the present disclosure highly express CD16 (over 70%), resolving the problem of low CD16 expression in existing iNK cells, which requires genetic modification to address;
8) The obtained iNK cells can be used to treat hematological tumors and solid tumors; and
9) iNK can be loaded with a CAR to perform specific killing.

Additional aspects and advantages of the present disclosure will be provided in part in the following description, or will become apparent in part from the following description, or can be learned from practicing of the present disclosure.

### BRIEF DESCRIPTION OF THE DRAWINGS

The foregoing and/or additional aspects and advantages of the present disclosure will become apparent and readily appreciated from the following description of the embodiments in conjunction with the accompanying drawings.

FIG. 1 shows a schematic diagram of a process of differentiation of iPSCs into HSPCs according to one embodiment of the present disclosure. The culture flask or culture bag in the figure generally refers to a culture vessel.

FIG. 2 shows a schematic diagram of a process of differentiation of iPSCs into iNK cells according to one embodiment of the present disclosure. The dashed lines represent steps in which different culture systems can be flexibly selected based on production capacity requirements. The culture flask or culture bag in the figure generally refers to a culture vessel.

FIG. 3 shows the proportion of CD34⁺CD43⁺ cells in the HSPC population differentiated from iPSCs in Example 1 of the present disclosure. In the classical method, the first differentiation medium is not supplemented with BMP4 or CHIR99021, and the second differentiation medium is not supplemented with UM171, CHIR99021, or SR1.

FIG. 4 shows the proportion of CD34⁺CD44⁺ cells in the HSPC population differentiated from iPSCs in Example 1 of the present disclosure.

FIG. 5 shows statistical results of the purity of CD34⁺ (HSPCs) differentiated from five iPSC lines in Example 1 of the present disclosure. iPSC-1 includes two independent experiments.

FIG. 6 shows statistical results of the fold expansion of CD34⁺ (HSPCs) differentiated from five iPSC lines in Example 1 of the present disclosure. iPSC-1 includes two independent experiments.

FIG. 7 shows the purity of iNK cells (>Day 40) obtained by the method of the present disclosure as detected by FACS in Example 1 of the present disclosure.

FIG. 8 shows CD16 expression of iNK cells (>Day 40) obtained by the method of the present disclosure as detected by FACS in Example 1 of the present disclosure.

FIG. 9 shows the proportion of iNK cells expressing activating receptors or co-stimulatory factors (>Day 40) obtained by the method of the present disclosure as detected by FACS in Example 1 of the present disclosure. Along the horizontal axis, the peak on the left represents isotype control, and the peak on the right represents the activating receptors or co-stimulatory factors.

FIG. 10 shows the fold expansion curve of iPSCs differentiating into iNK cells in Example 1 of the present disclosure.

FIG. 11 shows OCT4 expression on Day 3 of pluripotent stem cells differentiating into HSPCs in Example 2 of the present disclosure.

FIG. 12 shows the expression of early mesoderm marker MIXL1 and mesoderm marker T on Day 3 of pluripotent stem cells differentiating into HSPCs in Example 2 of the present disclosure.

FIG. 13 shows the expression of hemogenic endothelial progenitor cell markers KDR and SCL on Day 3 of pluripotent stem cells differentiating into HSPCs in Example 2 of the present disclosure.

FIG. 14 shows the expression of HSPC marker CD34 on Day 3 of pluripotent stem cells differentiating into HSPCs in Example 2 of the present disclosure.

FIG. 15 shows the effects of adding CHIR99021 and/or SR1 to the second differentiation medium on the proportion and yield of CD34⁺ or CD43⁺ cells in Example 3 of the present disclosure. A: Proportion of CD34⁺ cells at week 2 of differentiation when CHIR99021 was added to the second differentiation medium; B. Fold expansion of the cell population at week 2 of differentiation when CHIR99021 was added to the second differentiation medium; C. Proportion of CD34⁺ and CD43⁺ cells at week 2 of differentiation when SR1 was added to the second differentiation medium.

FIG. 16 shows the effects of adding UM171 to the second and/or third differentiation medium on the proportion of CD34⁺ and CD56⁺ cells and on the fold expansion during the differentiation in Example 3 of the present disclosure. A: Proportion of CD34⁺ cells at week 2 and proportion of CD56⁺ cells at week 5; B. Fold expansion of the cells at week 2 and week 5.

FIG. 17 shows the purity and fold expansion of CD56⁺ iNK cells when serum substitute, human AB serum, or fetal bovine serum (FBS) was used in the fourth differentiation medium in Example 4 of the present disclosure. Each triangle, square, or circle represents an independent experiment.

FIG. 18 shows the effects of adding an SPHK2 inhibitor to the second and third differentiation media on the purity and yield of HSPCs and iNK cells in Example 5 of the present disclosure.

FIG. 19 shows the effects of adding NAD⁺ to the fourth differentiation medium on the differentiation efficiency and yield of iNK cells in Example 5 of the present disclosure.

FIG. 20 shows the effects of adding HLA-C to the fourth differentiation medium on the differentiation efficiency and yield of iNK cells in Example 5 of the present disclosure.

FIG. 21 shows the effects of adding IL-2 or SB203580 to the fourth differentiation medium on the differentiation efficiency and yield of iNK cells in Example 5 of the present disclosure.

FIG. 22 shows the morphology of adherent iPSCs under a light microscope after co-co-culture of cord blood-derived NK (CB-NK) cells or iNK cells with iNK-derived iPSCs for 4 hours or 24 hours in Example 6 of the present disclosure.

FIG. 23 shows the post-thaw cell viability of iNK cells obtained after cryopreservation with several cryopreservation solutions in Example 10 of the present disclosure.

FIG. 24 shows the killing effect of thawed iNK cells obtained after cryopreservation with cryopreservation solution 3 against hematological tumor cells (K562, Forage, U937) and solid tumor cells (A549) under a microscope in Example 10 of the present disclosure. K562 is a chronic myeloid leukemia cell line, Forage is a diffuse large cell non-Hodgkin's lymphoma cell line, U937 is a human histiocytic lymphoma cell line, and A549 is a human non-small cell lung cancer cell line.

FIG. 25 shows the number of remaining target cells (measured by fluorescence intensity of the target cells) after co-culture for more than 40 hours of thawed iNK cells obtained after cryopreservation with cryopreservation solution 3 with U-87 MG-GFP (brain glioma cell line) as monitored in real time using Incucyte in Example 10 of the present disclosure, thereby obtaining the killing activity of iNK cells against U-87 MG cells.

FIG. 26 shows the specific tumor-killing activity of CAR-iNK cells differentiated from iPSCs in Example 11 of the present disclosure. A: Process of preparing CAR-iNK cells, with CAR introduced at the iPSC stage; B: Killing assay of CB-NK, WT iNK, and CAR-BCMA iNK against K562 (BCMA-negative); C: Comparison of the killing ability of CAR-BCMAiNK against H929 (a BCMA-high tumor cell line) with two other NK cell types; D: Dose-dependent killing effect of CAR-BCMA iNK against H929.

### DETAILED DESCRIPTION

The present disclosure is described below with reference to specific embodiments. It should be noted that these embodiments are merely illustrative and do not limit the present disclosure in any way.

Unless otherwise specified, all reagents used in the experiments of the examples can be obtained commercially.

Furthermore, the terms “first” and “second” are used for descriptive purposes only and are not to be construed as indicating or implying relative importance or implicitly indicating the number of technical features indicated. Thus, a feature defined as “first” or “second” may explicitly or implicitly include at least one such feature. In the description of the present disclosure, the meaning of “plurality” or “more” is at least two, e.g. two, three, etc. unless specifically and specifically limited otherwise.

The endpoints of the ranges and any values disclosed herein shall not limited to the exact range or value, and those ranges or values should be understood to include values close to those ranges or values. For numerical ranges, endpoints of the respective ranges, an endpoint of respective ranges and an individual point value, and individual point values may be combined with each other to obtain one or more new numerical ranges, which should be deemed to be specifically disclosed herein.

In order to make the present disclosure more readily understood, certain technical and scientific terms are specifically defined below. Unless explicitly defined otherwise elsewhere herein, all other technical and scientific terms used herein have the same meaning as commonly understood by one of ordinary skill in the art to which the present disclosure belongs.

As used herein, the terms “include” or “comprise” are open expressions, that is, including the contents specified in the present disclosure, but not excluding other contents.

### Terms

Closed system: A system designed and operated to prevent products or materials from being exposed to the room environment. When the products or materials are transferred into the closed system, they must be transferred in a non-exposed manner (e.g., through a sterile connector or a sealed transfer system) to avoid exposure of the products or materials to the room environment. If the closed system needs to be opened (e.g., for filter installation or connection), it must be disinfected or sterilized before returning to the closed state or prior to use.

3D continuous culture: A culture method in which cells are cultured using a non-adherent method, generally in suspension culture, without involving cryopreservation or thawing operations between different culture stages.

Shaking culture: A culture method in which specific equipment or culture vessels are used to allow the cell culture medium to move regularly or irregularly during the culture process of the cells.

Static culture: A culture method in which the culture medium remains substantially stationary relative to the culture vessel during the culture process of the cells.

Pluripotent stem cells: A type of stem cell with self-renewal ability and multi-lineage differentiation potential, expressing OCT4, NANOG, SSEA-4, and Tra-1-60. Representative examples include human induced pluripotent stem cells (iPSCs) and human embryonic stem cells (ESCs). The embryonic stem cells used herein are commercially available embryonic stem cells.

Pluripotent stem cells in a single-cell state: Pluripotent stem cells that have been dissociated from a clonal or spheroid state into single cells by specific enzymatic digestion.

CD34⁺ cells or HSPCs: Both types of cells refer to hematopoietic stem/progenitor cells, which can be obtained by primary isolation or differentiated from pluripotent stem cells, such as human iPSCs or ESCs, and which possess characteristics of hematopoietic stem/progenitor cells.

Spheroid: Cell aggregates formed by multiple cells in a shaking or static culture state, which have a relatively dense structure and require specific enzymatic digestion to dissociate into a single-cell state.

Pluripotent stem cell maintenance medium: a medium used to maintain the pluripotency of pluripotent stem cells and enable their proliferation.

Inhibitor: A substance that can specifically block a specific signaling pathway, generally a small-molecule compound, nucleic acid, protein, etc.

Serum substitute: A serum-free additive commonly used as a component of cell culture media to support survival or proliferation of the cells.

Chimeric antigen receptor: abbreviated as CAR, a receptor including an extracellular antigen-binding domain, typically in the form of a single-chain antibody connected by a flexible hinge region, a transmembrane domain, and an intracellular signaling domain.

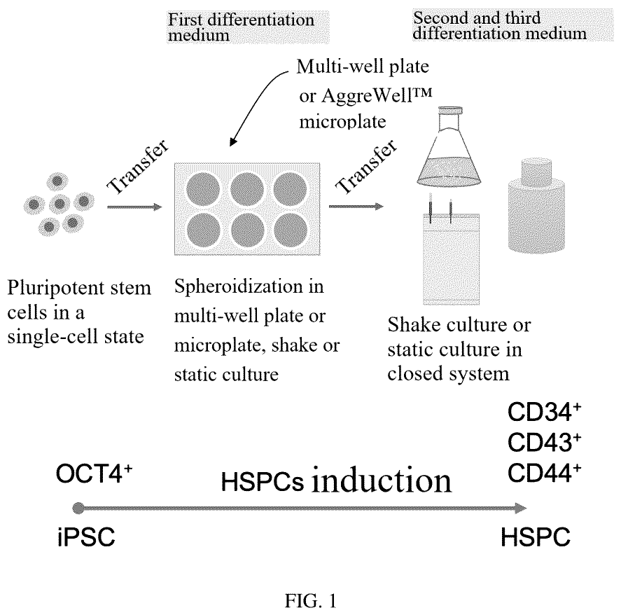

According to a specific embodiment of the present disclosure, the present disclosure provides a method for preparing CD34⁺ hematopoietic stem/progenitor cells. As shown in FIG. 1, the method includes:
A. obtaining pluripotent stem cells in a single-cell state;
B. culturing the pluripotent stem cells in the single-cell state under a first culture condition, to obtain a first cell spheroid;
C. culturing the first cell spheroid under a second culture condition, to obtain a second cell spheroid;
D. culturing the second cell spheroid under a third culture condition, to obtain the CD34⁺ hematopoietic stem/progenitor cells.

The first culture condition includes inducing differentiation of the pluripotent stem cells using a first differentiation medium.

The second culture condition includes inducing differentiation of the first cell spheroid using a second differentiation medium.

The third culture condition includes inducing differentiation of the second cell spheroid using a third differentiation medium.

The pluripotent stem cells are iPSCs or commercially available embryonic stem cells.

According to a specific embodiment of the present disclosure, (1) the first differentiation medium includes a first basal medium and BMP4 and a GSK-3β inhibitor;
(2) the second differentiation medium includes a second basal medium and optionally UM171, and does not include IL-3;
(3) the third differentiation medium includes a third basal medium, VEGF, bFGF, SCF, FLT3L, and TPO, and does not include TL-3.

According to a specific embodiment of the present disclosure, the pluripotent stem cells are iPSCs or commercially available embryonic stem cells.

According to a specific embodiment of the present disclosure, the first basal medium includes at least one selected from mTeSR™1, TeSR™2, TeSR™-AOF, Essential 8™ Culture Media, NutriStem® hESC XF, or StemFit® Feeder-Free Stem Cell Culture Media, preferably the first basal medium is mTeSR™1 or TeSR™-AOF medium; the second basal medium is StemPro™-34 SFM complete medium; and the third basal medium is StemPro™-34 SFM complete medium.

According to a specific embodiment of the present disclosure, the first differentiation medium further includes a Rock inhibitor.

The Rock inhibitor includes but is not limited to Y27632 and HB-100, and may also be other types of Rock inhibitors except Y27632 and HB-100.

According to a specific embodiment of the present disclosure, the second differentiation medium further includes VEGF, bFGF, and BMP4; the second differentiation medium further includes an induction enhancer; the induction enhancer includes at least one selected from a GSK-3β inhibitor or SR1; and the GSK-3β inhibitor includes at least one selected from CHIR99021, NP031112, AT7519, TWS119, SB216763, CHIR-98014, AZD1080, SB415286, LY2090314, (E/Z)-GSK-3β inhibitor 1, KY19382, Alsterpaullone, BIO-acetoxime, IM-12, 1-Azakenpaullone, or Indirubin.

According to a specific embodiment of the present disclosure, the second differentiation medium further includes ITS-X, β-mercaptoethanol, ascorbic acid, and GlutaMAX.

According to a specific embodiment of the present disclosure, the third differentiation medium further includes ITS-X, β-mercaptoethanol, ascorbic acid, and GlutaMAX.

According to a specific embodiment of the present disclosure, BMP4 is used at a concentration ranging from 5 ng/mL to 100 ng/mL;
CHIR99021 is used at a concentration ranging from 1 M to 20 M;
VEGF is used at a concentration ranging from 5 ng/mL to 100 ng/mL;
bFGF is used at a concentration ranging from 5 ng/mL to 100 ng/mL;
SCF is used at a concentration ranging from 5 ng/mL to 100 ng/mL;
TPO is used at a concentration ranging from 1 ng/mL to 100 ng/mL;
FLT3L is used at a concentration ranging from 1 ng/mL to 200 ng/mL, preferably from 1 ng/mL to 50 ng/mL;
UM171 is used at a concentration ranging from 0 nM to 1 μM;
SR1 is used at a concentration ranging from 0 μM to 2 μM.

The “used at a concentration” mentioned in the present disclosure is the final concentration used in the cell culture.

The method for obtaining pluripotent stem cells in a single-cell state may be performed by various approaches to allow subsequent aggregation into spheroids. For example, spheroids may be formed by shaking culture on an orbital shaker, or by centrifugation and subsequent static culture in an AggreWell™ microwell plate. In addition, a single-cell suspension of pluripotent stem cells may be obtained according to the method disclosed in Chinese Patent No. ZL201310276246.7, entitled “A method for passaging human induced pluripotent stem cells and use thereof” According to a specific embodiment of the present disclosure, a method for obtaining pluripotent stem cells in a single-cell state specifically includes the following steps. 1) For iPSCs or ESCs under culture, when the cell confluence reaches 75% to 85%, or when the culture time of the cell spheroids by 3D culture reaches 4 to 7 days, the cells are washed once with DMEM/F12 medium, added Accutase digestion enzyme or TrypLE digestion enzyme, and placed in a 37° C., 5% CO₂ incubator for digestion. When the cells are substantially dissociated, pluripotent stem cell maintenance medium (mTeSR™1) is added to terminate the digestion. 2) The system is centrifuged, the supernatant is discarded, and the cells are resuspended in mTeSR™1 or TeSR™-AOF medium supplemented with 5 μM ROCK inhibitor, 50 ng/mL BMP4, and 10 μM CHIR99021. As a result, iPSCs or ESCs in a single-cell state is prepared.

According to a specific embodiment of the present disclosure, the present disclosure provides a method for differentiating pluripotent stem cells into CD34⁺ cells, which takes about 6 to 14 days in total. The method includes the following consecutive steps:
1) In a pluripotent stem cell maintenance medium (first differentiation medium) containing BMP4, a GSK-3β inhibitor (e.g., CHIR99021), and a Rock inhibitor, multiple pluripotent stem cells in a single-cell state are aggregated for 12 to 48 hours to obtain the first spheroid. The spheroidization of the pluripotent stem cells can be performed by shaking culture in multi-well plates or by static culture in AggreWell™ microwell plates.
2) In a second differentiation medium containing BMP4, VEGF, bFGF, UM171, and an induction enhancer but lacking IL-3, the first spheroid is cultured for 1 to 7 days to obtain a second spheroid. During this period, the cells may optionally be transferred into a closed system. The closed system may be a culture bottle or a culture bag, and the culture method may be shaking culture or static culture. The induction enhancer includes at least one of the following components: a GSK-3β inhibitor, or SR1, or a combination thereof.
3) In a third differentiation medium containing VEGF, bFGF, SCF, FLT3L, and TPO but lacking IL-3, the second spheroid is cultured for 2 to 8 days to obtain the third spheroid and suspension cells.

According to a specific embodiment of the present disclosure, the pluripotent stem cell maintenance medium is a medium that can maintain the pluripotency of pluripotent stem cells. The GSK-3β inhibitor may be CHIR99021; and the Rock inhibitor may be Y27632, HB-100, or other substances that can support the survival of pluripotent stem cells in a single-cell state.

According to a specific embodiment of the present disclosure, BMP4 is used at a concentration ranging from 5 ng/mL to 100 ng/mL, CHIR99021 is used at a concentration ranging from 1 uM to 20 uM, VEGF is used at a concentration ranging from 5 ng/mL to 100 ng/mL, bFGF is used at a concentration ranging from 5 ng/mL to 100 ng/mL, UM171 is used at a concentration ranging from 0 nM to 1 uM, SR1 is used at a concentration ranging from 0 μM to 2 μM, SCF is used at a concentration ranging from 5 ng/mL to 100 ng/mL, TPO is used at a concentration ranging from 1 ng/mL to 100 ng/mL, and FLT3L is used at a concentration ranging from 1 ng/mL to 200 ng/mL.

By using the above-mentioned method, the number of CD34⁺ cells obtained at differentiation day 10 to day 14 can increase up to 99-fold (average approximately 72-fold) relative to the initial number of pluripotent stem cells, and the purity of CD34⁺ cells can reach up to 99% (average 94%), in the form of suspended HSPCs. Furthermore, the above HSPCs concurrently express CD43 or CD44. In a recent study (Zhu, Yanling et al. “Characterization and generation of human definitive multipotent hematopoietic stem/progenitor cells.” Cell discovery vol. 6, 1 89. 1 Dec. 2020), CD44 can be used as a marker to distinguish two different types of hematopoietic stem cells (primitive hematopoiesis and definitive hematopoiesis) in the early stages of human hematopoietic development, and can be used to identify definitive hematopoietic HSPCs generated from human pluripotent stem cells. CD44⁺ multipotent hematopoietic stem cells generated from human pluripotent stem cells exhibit multiple potentials, and can produce a variety of blood cells in vitro and in vivo, such as myeloid (My), erythroid (Er), and megakaryocytes (Mk), as well as more critical immune cells such as NK cells and T cells, thus having the potential to treat diseases. Therefore, the CD34⁺ cell population obtained by this method exhibits characteristics of definitive HSPCs derived from human pluripotent stem cells.

In some embodiments, the above-mentioned culture medium composition can be used to promote the differentiation of pluripotent stem cells into HSPCs. The first differentiation medium includes BMP4, a GSK-3β inhibitor, and a Rock inhibitor, as well as a medium capable of maintaining the pluripotency of the pluripotent stem cells. If animal component-free conditions are preferred, the culture medium used to maintain the pluripotency of the pluripotent stem cells include but are not limited to mTeSR™1, TeSR™2, TeSR™-AOF, Essential 8™ Culture Media, NutriStem® hESC XF, and StemFit® Feeder-Free Stem Cell Culture Media. Any of the above media or their equivalents can be used in this method. In the production of clinical application products, culture media that are free of animal components can be selected from the above. The equivalent used here refers to a medium that, when used alone or supplemented with only a ROCK inhibitor, is capable of maintaining pluripotent stem cells in a pluripotent state for more than 3 consecutive days. Specifically, the proportion of OCT4⁺ cells in the cultured cell population detected by flow cytometry shows a variation of no more than 20%.

In some embodiments, the addition of at least one of BMP4 or a GSK-3β inhibitor during the formation of the first spheroid of pluripotent stem cells significantly accelerates the differentiation of pluripotent stem cells into HSPCs during the subsequent differentiation process. By day 3 of differentiation, OCT4 expression significantly decreases, the expression of early mesoderm and mesodermal markers (MIXL1 and T) also begins to decrease, while the expression of hemogenic endothelial lineage-related markers (KDR, SCL) increases significantly. More notably, the expression of HSPC marker (CD34) significantly increases. At the beginning of this stage, the addition of an GSK-3β inhibitor and BMP4 to the pluripotent stem cell maintenance medium substantially accelerates the differentiation process, allowing detection of CD34 expression on day 3 of differentiation, with hematopoietic marker expression being significantly higher compared to control conditions.

In some embodiments, the concentrations of ITS-X, β-mercaptoethanol, ascorbic acid vitamin C, and 1× GlutaMAX contained in the second differentiation medium and the third differentiation medium may be within the conventional concentration ranges used in culture media for the differentiation of pluripotent stem cells into HSPCs known in the art. In some embodiments, the second differentiation medium and the third differentiation medium both include StemPro™-34 SFM complete medium, 0.1% to 5% ITS-X, 0.1 uM to 10 uM 0-mercaptoethanol, 5 μg/mL to 100 μg/mL ascorbic acid, and 1× GlutaMAX, and do not include IL-3. The second differentiation medium includes UM171, and the addition of UM171 can significantly increase the yield of CD34⁺ cells. Unlike previous methods in which UM171 is added throughout the entire HSPC differentiation process (Li, Xuejia et al. “Pyrimidoindole derivative UM171 enhances derivation of hematopoietic progenitor cells from human pluripotent stem cells.” Stem cell research vol. 21 (2017): 32-39.), in the differentiation system of the present disclosure, UM171 is added only to the second differentiation medium, while still achieving high CD34⁺ differentiation efficiency and optimal HSPC yield. In some embodiments, the second culture medium further includes at least one component of an induction enhancer to obtain a higher number or higher proportion of CD34⁺ cells. In some embodiments, the duration of action of the above three stages of media may affect the yield and proportion of CD34⁺ cells.

To avoid ambiguity, CD34⁺ cells, HSPC, or HSPCs used herein all refer to hematopoietic stem/progenitor cells, or a type of cells with hematopoietic stem/progenitor cell properties that can be differentiated from pluripotent stem cells (such as human iPSCs or ESCs). Such cells can be further differentiated into a variety of hematopoietic lineage cells, such as myeloid (My), erythroid (Er), and megakaryocytes (Mk), as well as lymphoid cells, such as NK cells, T cells, B cells (including plasma cells), etc. The pluripotent stem cells according to the present disclosure express OCT4, NANOG, SSEA-4, and Tra-1-60, and representative examples thereof are human induced pluripotent stem cells (iPSCs) and human embryonic stem cells (ESCs).

Furthermore, single or multiple CD34⁺ cells or HSPCs can be further differentiated into myeloid (My), erythroid (Er), and megakaryocytes (Mk), as well as lymphoid cells. Myeloid cells include myeloid progenitor cells, monocytes, macrophages, neutrophils, basophils, eosinophils, erythrocytes, megakaryocytes/platelets, and dendritic cells. Lymphoid cells include NK cells, T cells, and B cells (including plasma cells).

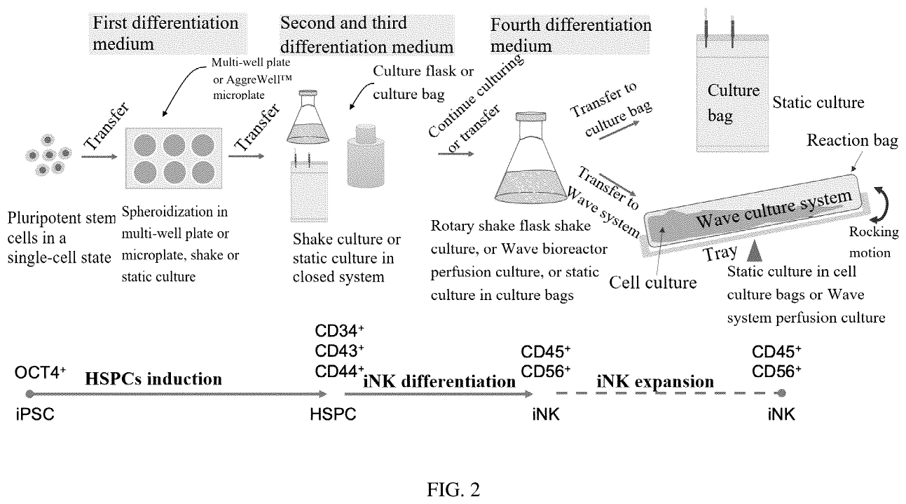

According to a specific embodiment of the present disclosure, the present disclosure provides a method for preparing NK cells. As shown in FIG. 2, the method includes:
a. obtaining CD34⁺ hematopoietic stem/progenitor cells using the above-mentioned method;
b. culturing the CD34⁺ hematopoietic stem/progenitor cells under a fourth culture condition, to obtain the NK cells.

The fourth culture condition includes inducing differentiation of the CD34⁺ hematopoietic stem/progenitor cells using a fourth differentiation medium, and
the second and/or the third differentiation medium further includes an SPHK2 inhibitor.

According to a specific embodiment of the present disclosure, the fourth differentiation medium includes a fourth basal medium, IL-7, IL-15, FLT3L, SCF, and optionally IL-3.

According to a specific embodiment of the present disclosure, the pluripotent stem cells are iPSCs or commercially available embryonic stem cells.

According to a specific embodiment of the present disclosure, the fourth basal medium may be DMEM/F12; and the SPHK2 inhibitor may be ABC294640, and ABC294640 is used at a concentration ranging from 1 M to 50 μM.

Optionally, IL-3 is used at a concentration ranging from 0 ng/mL to 10 ng/mL;
IL-7 is used at a concentration ranging from 0.1 ng/mL to 30 ng/mL;
IL-15 is used at a concentration ranging from 1 ng/mL to 10 ng/mL;
FLT3L is used at a concentration ranging from 1 ng/mL to 200 ng/mL, preferably from 1 ng/mL to 50 ng/mL;
SCF is used at a concentration ranging from 5 ng/mL to 100 ng/mL.

According to a specific embodiment of the present disclosure, the fourth differentiation medium further includes human serum albumin and at least one of serum substitute, human AB serum, or FBS.

In some embodiments, the fourth differentiation medium further includes serum substitute and human serum albumin. The dosage of serum substitute and human serum albumin can be within the conventional dosage range of serum substitute and human serum albumin contained in the differentiation medium used to induce differentiation of HSPCs into NK cells, for example, 2% serum substitute-3+0.2% to 1% human serum albumin. According to a specific embodiment of the present disclosure, the serum substitute may be any brand of serum substitute known in the art, for example, BIT 9500, SR3, T-NK Xeno-Free Serum Substitute, Knockout™ Serum Substitute, CTS™ Immune Cell Serum Substitute, etc.

According to a specific embodiment of the present disclosure, the fourth differentiation medium further includes at least one of NAD⁺, HLA-C, SB203580, or IL-2;
optionally, NAD⁺ is used at a concentration ranging from 1 μM to 500 μM;
HLA-C is used at a concentration ranging from 0.05 ng/mL to 1 ng/mL;
SB203580 is used at a concentration ranging from 1 M to 50 PM;
IL-2 is used at a concentration ranging from 100 IU to 5000 IU.

In some embodiments, the CD34⁺ cells obtained in this method can be cultured in a fourth differentiation medium to be differentiated into natural killer (iNK) cells. The iNK cells express CD56, CD45, CD16, NKG2D, NKp30, NKp44, NKp46, CD226, and 2B4, and highly expresses CD16. The components of the fourth differentiation medium include a basal medium and cytokines. The basal medium is DMEM/F12⁺ serum substitute+human serum albumin, wherein the serum substitute may be selected from at least one of BIT 9500, SR3, T-NK Xeno-Free Serum Substitute, Knockout™ Serum substitute, CTS™ Immune Cell Serum substitute, or any combination thereof. In some embodiments, the serum substitute in the fourth differentiation medium can also be substituted with human AB serum or FBS. The iNK cells with high CD56 expression obtained using serum substitute is similar to AB serum and superior to FBS. However, the yield of iNK cells obtained is more stable and reproducible, which is better than AB serum and FBS. In some embodiments, the cytokines in the fourth differentiation medium include IL-3, IL-7, IL-15, FLT3L, and SCF. In some embodiments, the cytokines in the fourth differentiation medium may not include IL-3. During this period, the cells are cultured in 3D conditions, using cell culture flasks or cell culture bags, and can be cultured with shaking culture or static culture. Industrially acceptable closed culture systems, such as wave bioreactors, can also be used. The concentrations of the above-mentioned cytokines in the fourth differentiation medium are as follows: IL3, 0 ng/mL to 10 ng/mL; IL-7, 0.1 ng/mL to 30 ng/mL; IL-15, 1 ng/mL to 10 ng/mL; FLT3L, 1 ng/mL to 200 ng/mL; and SCF, 5 ng/mL to 100 ng/mL. In the iNK cells (>Day 40) produced by this method, the CD56⁺CD45⁺ proportion is more than 99%, the CD16 expression is more than 70%, and multiple activating receptors and co-stimulatory factors are highly expressed. After about 5 weeks of differentiation, the number of the cells can increase by about 2000 times. In some embodiments, after the iNK cells obtained in this method are co-cultured with their donor iPSCs for 24 hours, under the condition of an effector-target ratio of 5:1, almost all cells are killed. In some embodiments, after the inventors cryopreserve the iNK cells obtained in this method, the viability of the thawed iNK cells under all tested cryopreservation conditions is above 80%, and the thawed live cell ratio under the optimal cryopreservation conditions can reach 96%. More importantly, the thawed cells still have good killing properties against solid tumors and blood tumors.

In some embodiments, the second differentiation medium includes UM171. The addition of UM171 can not only increase the yield of HSPCs, but also significantly increase the yield of iNK cells. In some embodiments, the addition of SPHK2 inhibitors to the second and third differentiation medium, while reducing the yield of HSPCs, can significantly increase the yield of iNK cells differentiated from these HSPCs. The SPHK2 inhibitor may be ABC294640, and its effective concentration may range from 1 M to 50 M. In some embodiments, at least one of the following components is added to the fourth differentiation medium: nicotinamide adenine dinucleotide (NAD⁺), HLA-C, SB203580, or IL-2, which can increase the yield of iNK cells without affecting the proportion of CD56⁺ or CD45⁺ cells. The concentration of NAD+ ranges from 1 M to 500 M, the concentration of HLA-C ranges from 0.05 ng/mL to 1 ng/mL, the concentration of SB203580 ranges from 1 μM to 50 μM, and the concentration of IL-2 ranges from 100 IU to 5000 IU.

In some embodiments, a chimeric antigen receptor (CAR), such as CAR-BCMA, is loaded at the iPSC stage, and these iPSCs can specifically kill target cells H929 (H929 expresses BCMA antigen) after being further differentiated into anti-BCMA CARNK cells using this method. The CAR includes an extracellular domain for antigen recognition, a transmembrane domain connected to the extracellular domain, and an intracellular domain connected to the transmembrane domain. In some embodiments, during the differentiation of HSPCs into iNK cells, as the suspension cells released from the culture system increase, a larger culture vessel can be used to increase the volume of culture medium for medium exchange. A person skilled in the art can readily adjust the vessel size based on the number of released suspension cells, thereby ensuring sufficient nutrient supply for the expanding cell in culture.

The present disclosure provides a method for preparing HSPCs, and such HSPCs can be further differentiated into NK cells, which have the ability to kill hematological tumors and solid tumors. Furthermore, by loading CAR at the iPSC stage, the iNK cells differentiated from the iPSCs have the ability to target tumor cells expressing specific antigens for killing.

In another aspect, the present disclosure discloses a pharmaceutical composition of HSPCs (and/or iPSCs) differentiated from pluripotent stem cells according to any embodiment.

In another aspect, the present disclosure discloses a pharmaceutical composition of NK cells (and/or iPSCs) differentiated from pluripotent stem cells or HSPCs according to any embodiment.

In another aspect, the present disclosure discloses a pharmaceutical composition of NK cells (and/or iPSCs) differentiated from CAR-loaded pluripotent stem cells according to any embodiment.

In another aspect, the present disclosure discloses a potential method for providing immune cell therapy to a subject suffering from a disease. Generally, the method includes administering the above-mentioned iNK cells or CAR-iNK cells to the subject. The iNK cells can kill hematological tumors or solid tumors, and the CAR-iNK cells can specifically kill tumor cells expressing specific antigens.

The present disclosure provides a method in which, during the embryoid body formation stage, the addition of BMP4 and a GSK-3β inhibitor (e.g., CHIR99021) into the pluripotent stem cell maintenance medium significantly increases the purity of CD34⁺, CD43⁺, and CD44⁺ cells, with the purity of CD44⁺ cells close to 100%. Without requiring any intermediate sorting or enrichment steps, more than 90% CD34⁺ cells can be stably differentiated from iPSCs within 2 weeks. The method can yield CD34⁺ cells, CD43 cells, and CD44⁺ cells with a purity of up to more than 99%, as well as CD34⁺/CD43⁺ cells of HSPCs differentiated from iPSCs exceeding 90%. Compared with the initial number of pluripotent stem cells, the number of CD34⁺ cells can reach an average 70-fold expansion (up to 99-fold). The iNK cells obtained by further differentiation exhibit very high purity and elevated expression of activating receptors and costimulatory factors. The iNK cells obtained by the present method highly expresses CD16 (more than 70%), which solves the problem of low iNK CD16 amount (only 20% to 30%) in the prior art that requires genetic modification to address the problem of low CD16 expression. The differentiation process is conducted under 3D culture conditions, compatible with a closed culture system. The yield of iNK cells is high, and a single iPSC can be differentiated into 2,000 NK cells. The obtained iNK cells can be used to treat hematological tumors and solid tumors. The iNK can be loaded with CAR and specifically kill tumor cells.

The embodiment of the present disclosure will be explained below with reference to examples. Those skilled in the art will understand that the following examples are only used to illustrate the present disclosure and should not be considered to limit the scope of the present disclosure. Where specific techniques or conditions are not specified in the examples, they are performed according to techniques or conditions described in the literature in the art or according to the product description. The reagents or instruments used are conventional products that can be obtained commercially without indicating the manufacturer.

### Example 1: Differentiation from iPSC to HSPCs and iNK Cells

### 1. Preparation of Pluripotent Stem Cells in a Single-Cell State

The preparation of pluripotent stem cells in a single-cell state can be carried out with reference to the method disclosed in Chinese Patent No. ZL201310276246.7 (“A method for passaging human induced pluripotent stem cells and its application”), to obtain a suspension of pluripotent stem cells in a single-cell state.

Specifically, the method includes the following steps: 1) For iPSCs in culture, when the cell confluence reached 75% to 85%, or when the culture time of the cell spheroids by 3D culture reached 4 to 7 days, the cells were washed once with DMEM/F12 medium, added Accutase digestion enzyme or TrypLE digestion enzyme, and placed in a 37° C., 5% CO₂ incubator for digestion. When the cells were substantially dissociated, pluripotent stem cell maintenance medium (mTeSR™1) was added to terminate the digestion. 2) The system was centrifuged, the supernatant was discarded, and the cells were resuspended in mTeSR™1 medium supplemented with 5 μM Y27632, 50 ng/mL BMP4, and 10 μM CHIR99021. As a result, iPSCs in a single-cell state was prepared.

2. Differentiation of iPSCs into HSPCs

### (1) First Stage Differentiation of HSPCs

iPSCs were aggregated into spheroids by either static spheroidization or shaking spheroidization.
1) Static spheroidization. An appropriate number of iPSCs in a single-cell state were transferred to an AggreWell™ microwell plate, and subsequent operations were carried out according to the AggreWell™ 400 or AggreWell™ 800 instructions. Briefly, the concentration of the single-cell suspension was adjusted, and sufficient volumes of the single-cell suspension were added to each well of the AggreWell™ 400 or AggreWell™ 800 to achieve approximately 500,000 iPSCs in a single-cell state per well. Then, each well was supplemented with mTeSR™1 or TeSR™-AOF medium, and 5 μM Y27632 was added to reach a total of 5 mL/well. Then, a balance plate filled with water was prepared to match the weight and position of the AggreWell™ microwell plate in the centrifuge. The cells were then pipetted gently up and down several times to ensure uniform distribution of cells across the wells. The AggreWell™ plate was immediately centrifuged at 100 g for 3 minutes using the balanced plate prepared in the previous step to force the cells into the microwells. The plate was observed under a microscope to verify that the cells were evenly distributed in the microwells. The cells were cultured under static conditions at 37° C., 5% CO₂, and 95% humidity for 1 to 2 days.
2) Shaking spheroidization. The concentration of the single-cell suspension was adjusted, and 1.5 million iPSCs in a single-cell state were transferred to a 6-well plate. Each well was supplemented with mTeSR™1 medium containing 5 μM Y27632, 50 ng/mL BMP4, and 10 μM CHIR99021 to achieve a volume of 3 mL per well. The culture plate was placed on an orbital shaker at 60 rpm, 70 rpm, 80 rpm, or 90 rpm for culture, and the wells with uniform sized spheroids were selected for subsequent culture.

The proportions of CD34⁺ cells obtained by the above two methods is shown in Table 1 below.

### [Table 1] Effects of Orbital Shaker and AggreWell Spheroidization Methods on the Purity of CD34⁺ Cell Populations

| Initial cell number | | |
|---|---|---|
| (millions) | Spheroidization method | Day 10 to 14, CD34⁺ |
| 1.5 | Orbital shaker | 91.1% |
| 0.5 | Aggre Well ™800 | 90.4% |
| 0.2 to 0.5 | Aggre Well ™400 | 90.7% |

The results in Table 1 show that both the orbital shaker and AggreWell spheroidization methods can obtain CD34⁺ cell populations with high purity.

### (2) Second Stage Differentiation of HSPCs

After 1 to 2 days of cell spheroidization, the medium was replaced with a second differentiation medium, and then the medium was changed every 2 days for 4 to 5 days of culture. The components of the second differentiation medium included: StemPro-34 complete medium supplemented with 1% ITS-X, 1 μM β-mercaptoethanol, 50 μg/mL ascorbic acid, 1× GlutaMAX, 50 ng/mL VEGF, 50 ng/mL bFGF, 50 ng/mL BMP4, and 35 nM UM171. At this stage, the cells can be transferred to a closed system for static culture or shaking culture.

### (3) Third Stage Differentiation of HSPCs

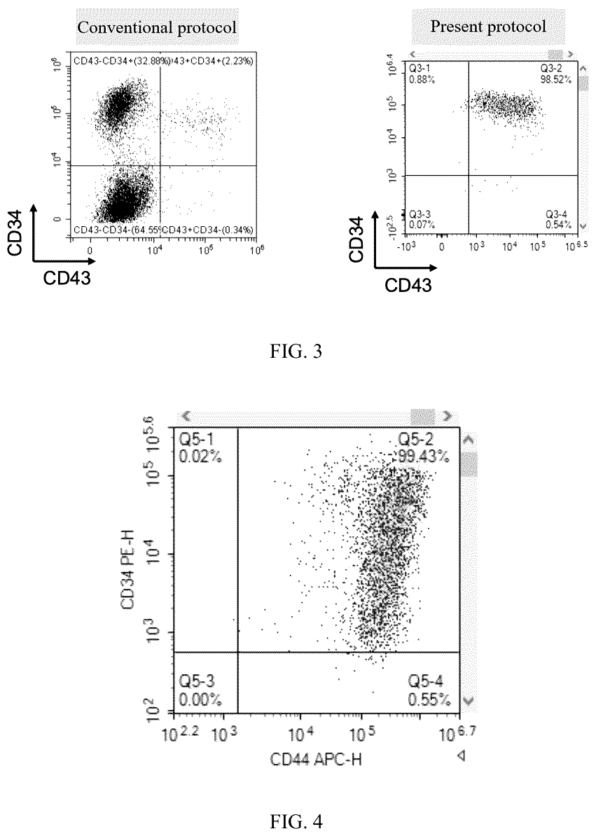

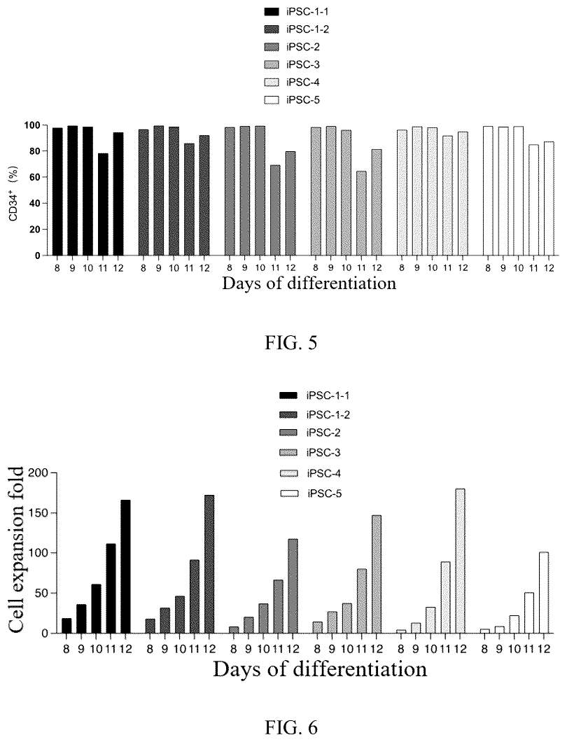

After culturing for 3 days in the second differentiation medium, the medium was replaced with a third differentiation medium. The components of the third differentiation medium included: StemPro-34™ SFM complete medium supplemented with 1% ITS-X, 1 μM β-mercaptoethanol, 50 μg/mL ascorbic acid, 1× GlutaMAX, 50 ng/mL VEGF, 50 ng/mL bFGF, 50 ng/mL SCF, 10 ng/mL FLT3L, and 30 ng/mL TPO. The expression of CD34 or CD43, and CD44 was detected by FACS. The CD34 antibody, the CD43 antibody, and the CD44 antibody were all purchased from Biolegend. The detection steps of FACS can be performed in reference to the commonly used operation specifications of the relevant instruments. The results are shown in FIG. 3 and FIG. 4, which indicate that the CD34⁺ cells differentiated by the method according to the present disclosure express both CD43 and CD44 concurrently. Meanwhile, the conventional method was used as a control, in which no BMP4 or CHIR99021 was added to the first differentiation medium, and no CHIR99021, or UM171, or SR1 was added to the second differentiation medium. Using this method, five iPSC cell lines, numbered iPSC-1, iPSC-2, iPSC-3, iPSC-4, and iPSC-5, were selected and differentiated into HSPCs. The proportion and fold expansion of CD34⁺ cells were detected at five time points: Day 8, Day 9, Day 10, Day 11, and Day 12. For iPSC-1, two independent experiments (iPSC-1-1, iPSC-1-2) were included. The results were shown in FIG. 5 and FIG. 6. It can be seen that the number of CD34⁺ cells obtained on Day 12 of differentiation increased by 100 to 180 fold relative to the initial pluripotent stem cell number, and the purity of CD34⁺ cells can reach 96% to 99% on Day 8. This result shows that the present disclosure has universal applicability.

(4) Differentiation of HSPCs into NK Cells

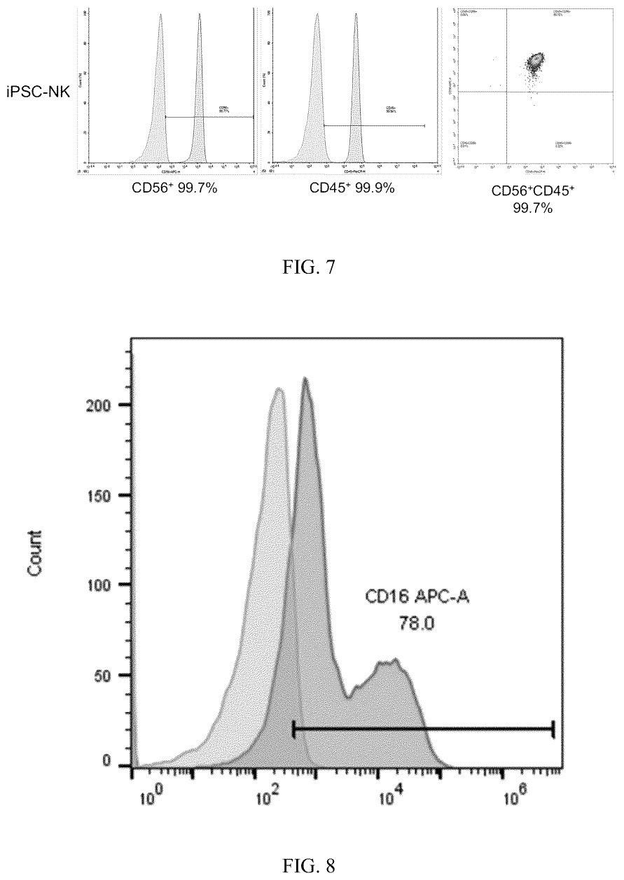

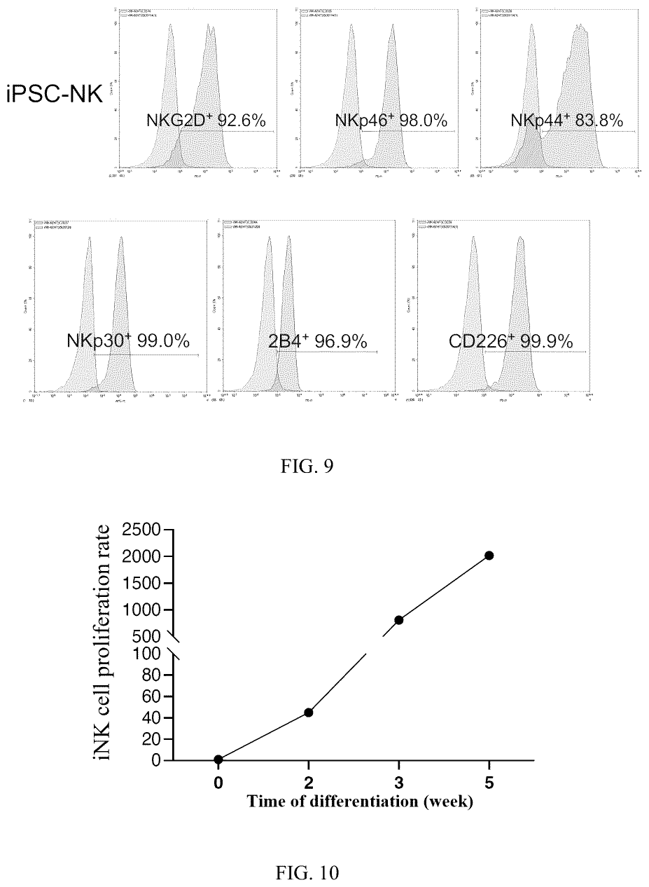

After three-stage differentiation of culture, generally on Day 10 to 14 of differentiation, depending on the FACS results, the culture medium was replaced with a fourth differentiation medium to further differentiate HSPCs into NK cells. This process generally lasted for 3 to 5 weeks. During differentiation and expansion, the expression of iNK markers CD45 and CD56 gradually increased, reaching a purity of over 99% after more than 40 days (FIG. 7). During this period, the expression level of CD16 also gradually increased, reaching more than 70% (FIG. 8). The fourth differentiation medium included a basal medium and cytokines. As an example, the basal medium was: DMEM/F12+2% serum substitute-3 (Sigma-Aldrich)+0.2% to 1% human serum albumin. The cytokines used included IL-3 (used in the first week), 20 ng/mL IL-7, 10 ng/mL IL-15, 10 ng/mL FLT3L, and 50 ng/mL SCF. The expression of CD56, CD45, CD16, NKG2D, NKp30, NKp44, NKp46, CD226, and 2B4 were detected by FACS. The antibodies (including isotype control) were all purchased from Biolegend. The detection steps of FACS can be performed in reference to the commonly used operation specifications of the relevant instruments. The results are shown in FIG. 7 to FIG. 9, which indicate that in the iNK cells (>Day 40) produced by the culture method according to the present disclosure, CD56⁺CD45⁺ proportion was over 99% (FIG. 7), CD16 expression was over 70% (FIG. 8), and multiple activating receptors and co-stimulatory factors were highly expressed (FIG. 9). After about 5 weeks of differentiation, the cell number can be expanded by about 2000-fold (FIG. 10).

### Example 2: Optimization of First Differentiation Medium

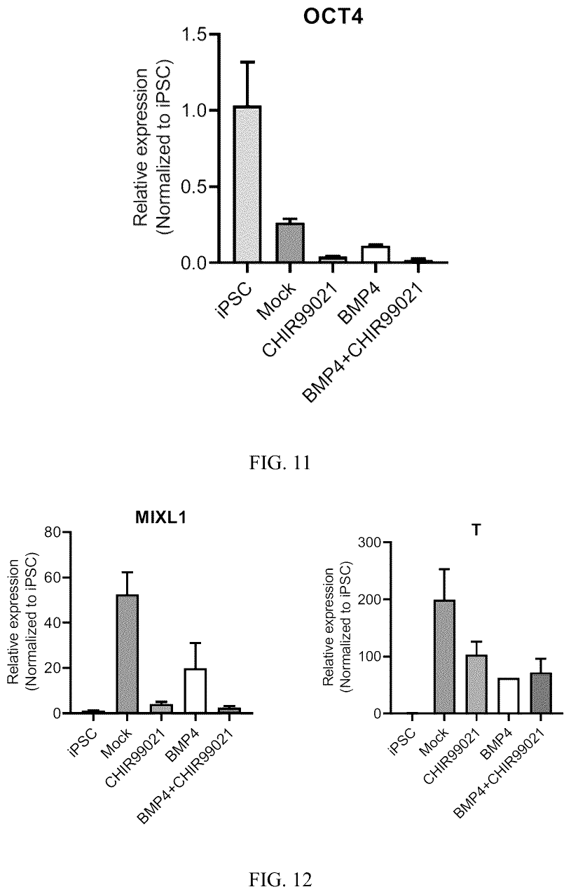

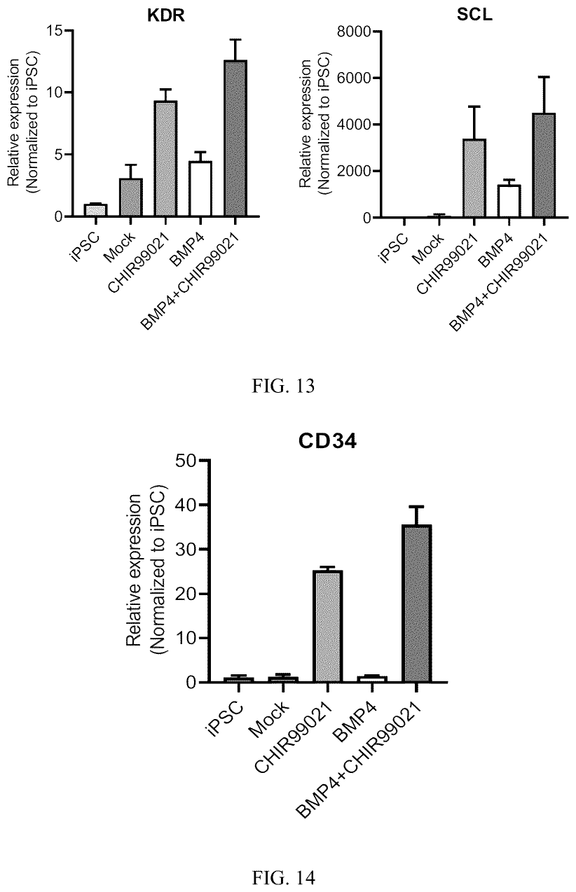

The iPSCs in the single-cell state were resuspended in the following four media after harvesting: 1) mTeSR™1+5 μM Y27632, 2) mTeSR™1+5 μM Y27632+50 ng/mL BMP4, 3) mTeSR™1+5 μM Y27632+10 μM CHIR99021, and 4) mTeSR™1+5 μM Y27632+50 ng/mL BMP4+10 μM CHIR99021. The cells were then cultured according to the method in Example 1. On Day 3 of differentiation, the cells were lysed, and mRNA was extracted and reverse-transcribed to obtain cDNA. The primers in Table 2 were used to measure the expression levels of OCT4, MIXL1, T, KDR, SCL, and CD34 in HSPCs on Day 3 of differentiation under the above-mentioned media conditions. The results are shown in FIG. 11 to FIG. 14, which indicate that on Day 3 of differentiation, the expression of OCT4 decreased significantly (FIG. 11), the expression of early mesoderm and mesoderm markers (MIXL1 and T) also began to decrease (FIG. 12), and the expression of hemogenic endothelial lineage-related markers (KDR, SCL) increased significantly (FIG. 13). More significantly, the expression of the HSPC marker (CD34) was significantly increased (FIG. 14). It shows that during the spheroidization stage of pluripotent stem cells, the addition of GSK-3β inhibitor and BMP4 to the pluripotent stem cell medium can substantially accelerate the differentiation process, and CD34 expression can be detected as early as Day 3 of differentiation. Moreover, the expression of hematopoietic system markers was significantly higher compared to control conditions, and the CD34 expression was significantly improved.

TABLE 2
qPCR primers

| | | SEQ |
|---|---|---|
| Gene | Primer (5′-3′) | ID NOS: |
| OCT4 | F1: AGTTTGTGCCAGGGTTTTTG | 1 |
| | R1: ACTTCACCTTCCCTCCAACC | 2 |
| KDR | F1: GTGATCGGAAATGACACTGGAG | 3 |
| | R1: CATGTTGGTCACTAACAGAAGCA | 4 |
| SCL | F1: CCAAAGTTGTGCGGCGTATC | 5 |
| | R1: CAGGCGGAGGATCTCATTCTT | 6 |
| CD34 | F1: CTACAACACCTAGTACCCTTGGA | 7 |
| | R1: GGTGAACACTGTGCTGATTACA | 8 |
| MIXL1 | F1: GGCGTCAGAGTGGGAAATCC | 9 |
| | R1: GGCAGGCAGTTCACATCTACC | 10 |
| T | F1: TATGAGCCTCGAATCCACATAGT | 11 |
| | R1: CCTCGTTCTGATAAGCAGTCAC | 12 |
| GAPDH | F1: GGAGCGAGATCCCTCCAAAAT | 13 |
| | R1: GGCTGTTGTCATACTTCTCATGG | 14 |

### Example 3: Analysis of Effect of Adding CHIR99021, SR1, or UM171 During Differentiation on HSPCs or iNK Cells

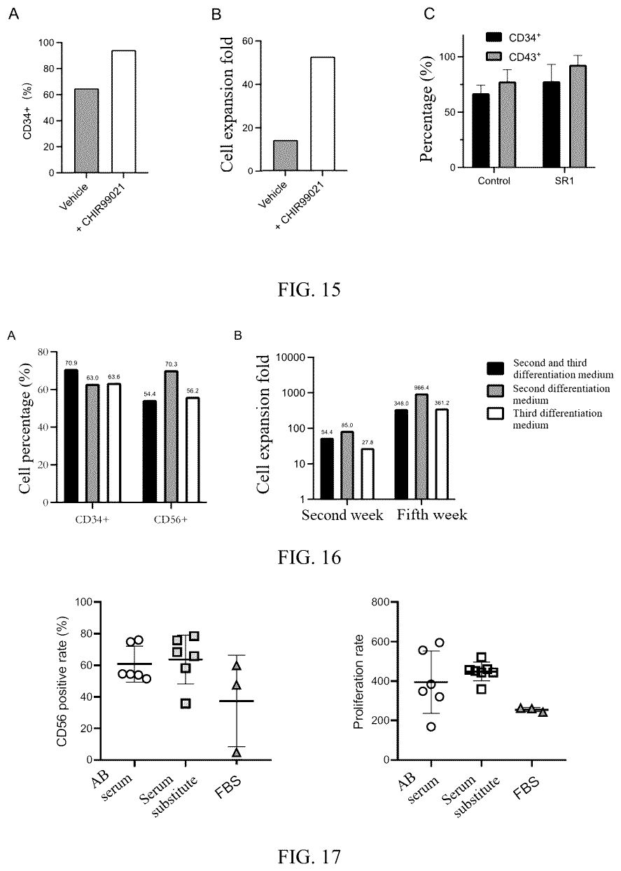

In order to further increase the yield and proportion of CD34⁺ cells, 10 μM CHIR99021 or 0.75 μM SR1 was added to the second differentiation medium of Example 1, and the proportion of CD34⁺ cells in the cell population on Day 12 was detected by FACS. The results are shown in FIG. 15. CHIR99021 and SR1 can increase the proportion of CD34⁺ cells in the cell population on Day 12.

35 nM UM171 was added to the second differentiation medium and/or the third differentiation medium in Example 1, and the proportion of CD34⁺ cells in the cell population on Day 12 and the proportion of CD56⁺ cells in the cell population on Day 35 were detected by FACS. The results at these two time points are shown in FIG. 16, which indicate that the addition of UM171 to the second differentiation medium can increase the yield of HSPCs differentiated from iPSCs and the yield of iNK cells differentiated from HSPCs.

### Example 4: Optimization of Serum Substitute in NK Cell Induction Medium

BIT 9500, SR3, T-NK Xeno-Free Serum Substitute, Knockout™ Serum Substitute, CTS™ Immune Cell Serum substitute, or a combination thereof (in FIG. 17, each square in the serum substitute group represents a serum substitute or combination from a different manufacturer, with a concentration ranging from 2% to 20% determined according to the manufacturer's instructions) were tested as controls. The performance of 15% human AB serum or 10% fetal bovine serum (FBS) in the induction of NK cells was also tested. The results are shown in FIG. 17, which shows that multiple serum substitutes supported efficient induction of HSPCs into NK cells, and the yield of iNK cells was slightly higher and more stable than that obtained with AB serum.

### Example 5: Optimization of iNK Cells Purity and Yield

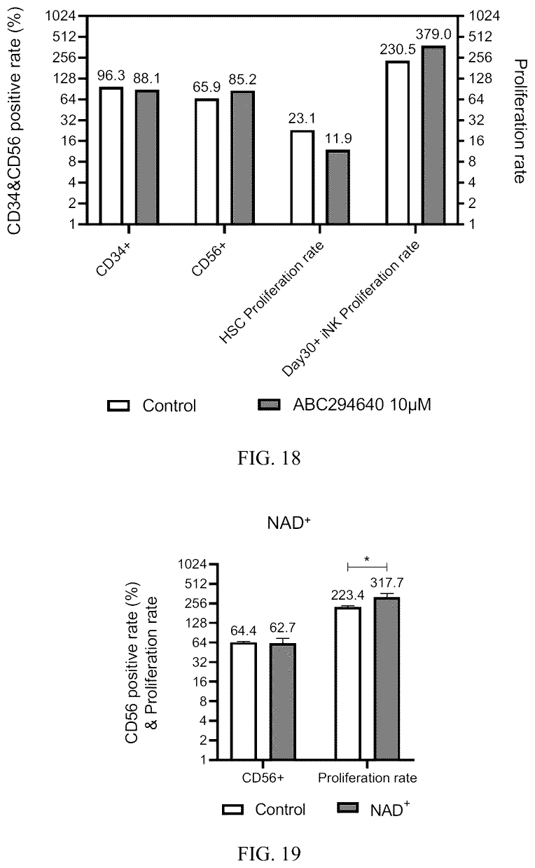

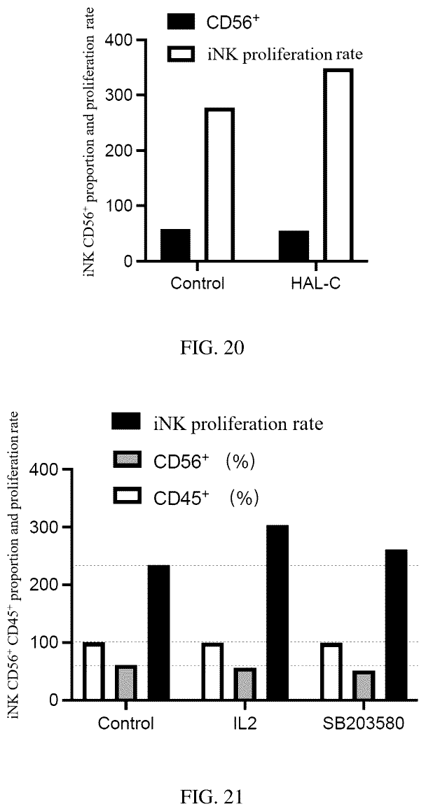

To further improve the differentiation efficiency and cytotoxicity of the obtained NK cells, UM171 was added to the second differentiation medium. Additionally, 1) 10 M SPHK2 inhibitor ABC294640 was added to the second and third differentiation medium, or 2) 75 M nicotinamide adenine dinucleotide (NAD⁺), or 0.1 ng/mL HLA-C, or 1000 IU IL-2, or 15 μM SB203580 was added to the fourth differentiation medium. The expression levels of CD56 and CD45 of the obtained NK cells and the cell fold expansion were measured by flow cytometry. The results are shown in FIG. 18 to FIG. 21. FIG. 18 shows that the addition of the SPHK2 inhibitor (ABC294640) to the second and third differentiation medium, while reducing the yield of HSPCs, can significantly increase the yield of iNK cells differentiated from these HSPCs. FIG. 19 to FIG. 21 shows that the addition of at least one of the following components to the fourth differentiation medium: nicotinamide adenine dinucleotide (NAD⁺), or HLA-C, or SB203580, or IL-2, can increase the yield of iNK cells without affecting the proportion of CD56⁺ or CD45⁺ cells. The results show that SPHK2 inhibitor, NAD, HLA-C, IL-2, and SB203580 can all increase the proliferation rate of iNK cells.

### Example 6: Killing Ability Assay of iNK Cells on Donor iPSCs

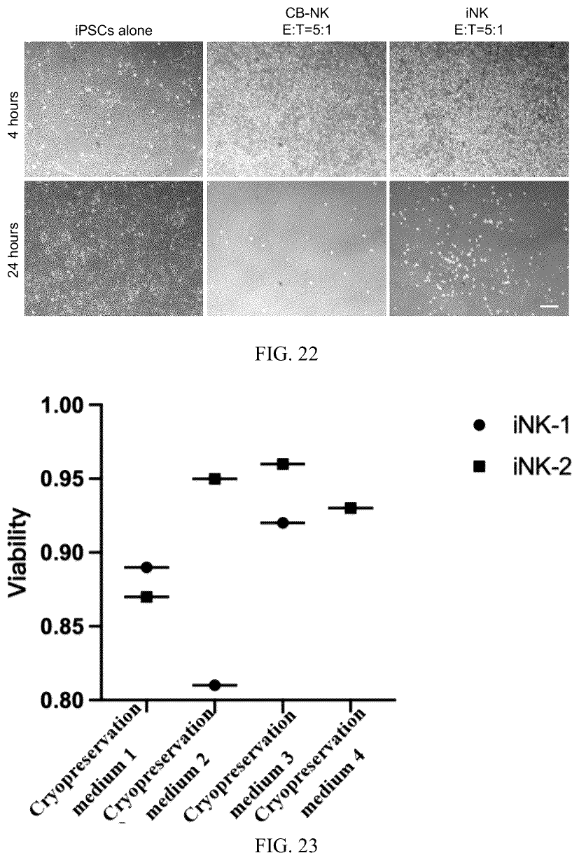

Using conventional methods, iPSCs were plated onto Matrigel-coated 6-well plate at a density of 250,000 per well. The concentration of Matrigel was used according to the manufacturer's instructions. On the first day of plating, the cells were cultured in mTeSR™1 medium supplemented with 5 μM Y27632. On the second day and thereafter, fresh mTeSR™1 medium was used for medium exchange. When the cells grew to 60% to 70% confluence, one well was harvested to count the iPSCs. Then, 5-fold amounts of CBNK or iNK cells were added to each well where iPSCs were cultured, depending on the number of iPSCs obtained by counting. Photographs were taken using an inverted microscope after 4 and 24 hours of co-culture. The results are shown in FIG. 22, which show that the iNK cells obtained by this method can eliminate their parental iPSCs within 24 hours under the condition of an effector-target ratio of 5:1.

### Example 7: Cell Expansion Calculation

The cell expansion at the HSPC stage and the NK stage was calculated by dividing the number of suspension cells obtained at the sampling time point by the initial number of iPSCs at the spheroidization stage. The cell number was determined using the Countess™ 2 automated cell counter, and specific operating procedures can be found in the instrument's instructions.

### Example 8: Killing Ability Assay of NK Cells Against Tumor Cells—Luciferase Method

Taking tumor cells K562-Luc (target cells) as an example, firstly, a target cell suspension (target cell viability was not less than 90%) was prepared and the cells were counted. The cells were then plated into the wells of a 96-well plate according to the specified effector-target ratio (e.g., 10:1, 5:1, 2:1, 1:1), with at least 3 replicate wells at each effector-target ratio. The medium in each well was adjusted to 100 μL, and then the required number of iNK cells or control NK cells were added according to the specified effector-target ratio. The 96-well plate was plated in a 37° C., 5% CO₂ incubator and incubated for the specified duration (e.g., 4 h or 24 h). The luciferase substrate was diluted 100-fold with the culture medium. 50 ul of the diluted luciferase substrate solution was added to each well, and the mixture reacted at room temperature for 10 minutes in the dark. The light source of the microplate reader was set to Lum and the time 1000 ms, and the plate was placed on the machine for measurement. Killing efficiency %=(control group−killing group)/cell control group*100.

### Example 9: Killing Ability Assay Against Tumor Cells—Incucyte Method

Taking tumor cells U-87 MG-GFP (target cells) as an example, firstly, a target cell suspension (target cell viability was not less than 90%) was prepared and the cells were counted. The cells were then plated into the wells of a 96-well plate according to the specified effector-target ratio (e.g., 10:1, 5:1, 2:1, 1:1), with at least 3 replicate wells at each effector-target ratio. The medium in each well was adjusted to 100 μL, and the cell adhesion was observed after 24 h to 48 h. Then, the required number of iNK cells or control NK cells were added according to the specified effector-target ratio. The 96-well plate was placed in an Incucyte© Live Cell Analysis System (Sartorius) in a 37° C., 5% CO₂ incubator, and images were acquired every 45 min or 1 h. After 24 h or 48 h, the cells were removed, the image data were retrieved, and the killing efficiency was calculated using Incucyte® software.

### Example 10: Cryopreservation and Thawing of iNK Cells

Cryopreservation of iNK cells: iNK cells were collected and centrifuged to remove the supernatant. The cells were resuspended in CS10 cryopreservation medium, or in other commercial cryopreservation mediums (cryopreservation mediums 1 to 3), or in homemade cryopreservation medium (iNK medium+10% DMSO, cryopreservation medium 4), allowing the cell density to be 25 million cells/mL. The cells were then aliquoted into cryovials at 1 mL per vial. The cryovials containing cells were quickly transferred into a programmed freezing container and placed in a −80° C. refrigerator overnight. On the next day, the cryovials were transferred to liquid nitrogen.

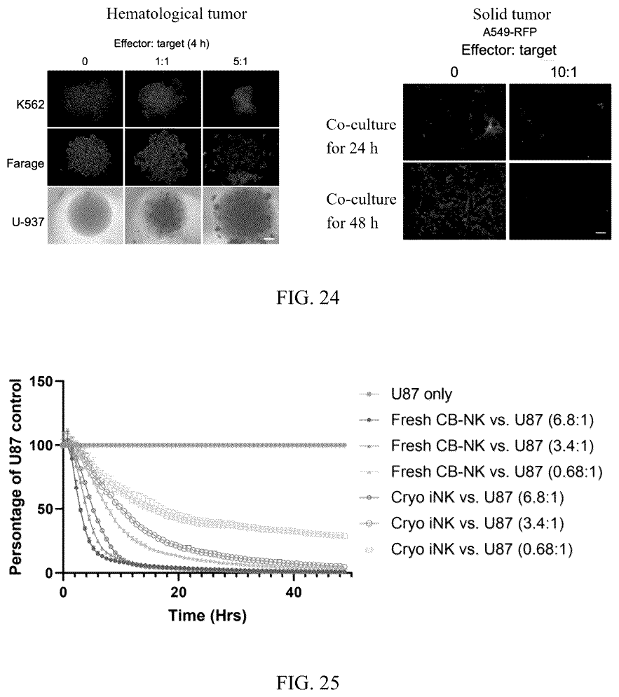

Thawing of iNK cells: The cryovials were removed from liquid nitrogen, rubbed with both hands a few times, and then quickly transferred to a 37° C. water bath or metal bath with constant shaking until only a small piece of ice remained in the cryovials. The outside of the cryovials were wiped quickly with 70% alcohol, and the cryovials were gently opened in the safety cabinet. The cell suspension was gently pipetted into a centrifuge tube in step 1 (shaking while adding dropwise) and mixed well. The cell suspension was mixed and then centrifuged (200 g*5 min). The supernatant was removed, and 10 mL of iNK medium was added again for resuspending and mixing. The cells were counted and then centrifuged. Cell inoculation: The supernatant was removed (be careful not to remove the cells). Depending on the cell number, cells were resuspended in NK medium at a cell density ranging from 1.0 million/mL to 1.6 million/mL, mixed well, and inoculated into culture flasks/culture plates. The cell viability of iNK cells after thawing from cryopreservation in several cryopreservation media is shown in FIG. 23, which indicates that the cell viability was well maintained after thawing from cryopreservation. The killing effects on hematological tumor or solid tumor cells are shown in FIG. 24 and FIG. 25, which indicates that the thawed iNK cells after cryopreservation had killing effects against both hematological tumor and solid tumor cells, and iNK cells have killing activity against U-87 MG cells.

### Example 11: Genetic Modification of iPSCs and CAR Introduction

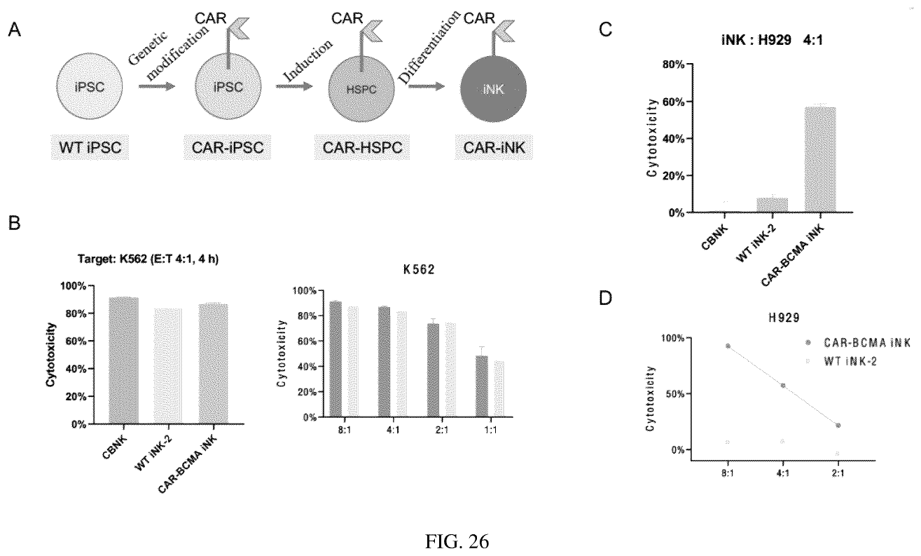

Anti-BCMA CAR was cloned into the piggybac vector. Subsequently, the piggybac plasmid expressing the anti-BCMA CAR and the plasmid expressing the transposase were co-transfected into a healthy human donor-derived iPSC line by nuclear transfection. CAR-BCMA iPSCs were differentiated and expanded into iNK cells using the method described in Example 1 (panel A in FIG. 26).

The complete amino acid sequence of CAR-BCMA is as set forth in SEQ ID NO: 15:

QVKLEESGGGLVQAGRSLRLSCAASEHTFSSHVMGWFRQAPGKERESVA
VIGWRDISTSYADSVKGRFTISRDNAKKTLYLQMNSLKPEDTAVYYCAA
RRIDAADFDSWGQGTQVTVSSGGGGSGGGGSGGGGSEVQLVESGGGLVQ
AGGSLRLSCAASGRTFTMGWFRQAPGKEREFVAAISLSPTLAYYAESVK
GRFTISRDNAKNTVVLQMNSLKPEDTALYYCAADRKSVMSIRPDYWGQG
TQVTVSSLINTTTPAPRPPTPAPTIASQPLSLRPEACRPAAGGAVHTRG
LDFACDSNLFVASWIAVMIIFRIGMAVAIFCCFFFPSWRRKRKEKQSET
SPKEFLTIYEDVKDLKTRRNHEQEQTFPGGGSTIYSMIQSQSSAPTSQE
PAYTLYSLIQPSRKSGSRKRNHSPSFNSTIYEVIGKSQPKAQNPARLSR
KELENFDVYSRVKFSRSADAPAYKQGQNQLYNELNLGRREEYDVLDKRR
GRDPEMGGKPRRKNPQEGLYNELQKDKMAEAYSEIGMKGERRRGKGHDG
LYQGLSTATKDTYDALHMQALPPR

On Day>40, iNK cells and target cells (K562-luc or H929-luc) were mixed at effector-target ratios of 8:1, 4:1, 2:1, and 1:1, and cultured at 37° C. 5% CO₂ for 4 hours. The killing of target cells by iNK cells was determined using the luciferase method (panels B to D in FIG. 26). The panel B shows the killing assay of CB-NK, WT iNK, and CAR-BCMA iNK against K562 cells (which do not express BCMA). The inherent killing ability of CAR-BCMAiNK against K562 at different effector-target ratios showed a dose-response effect and decreased with decreasing effector-target ratio. The panel C shows that CAR-BCMA iNK exhibits far superior killing ability against H929 (a tumor cell line that highly expresses BCMA) than the other two NK cells, suggesting that CAR-BCMA here can mediate the specific killing of tumor cells by iNK cells. The panel D shows that CAR-BCMAiNK exhibits a dose-response effect in killing H929 cells. These results demonstrate that after loading chimeric antigen receptor (CAR-BCMA) at the iPSC stage, these iPSCs can specifically kill the target cells H929 (H929 expresses BCMA antigen) after being further differentiated into anti-BCMA CAR-NK using the culture method according to the present disclosure.

In the specification, the description of the reference terms such as “one embodiment”, “some embodiments”, “example”, “specific example”, “some implementation methods”, or “some examples” means that the specific features, structures, materials, or characteristics described with reference to the embodiment or example are included in at least an embodiment or example of the present disclosure. In this specification, exemplary descriptions of the foregoing terms do not necessarily refer to the same embodiment or example. Moreover, the specific features, structures, materials, or characteristics described may be combined in any one or more embodiments or examples in a suitable manner. Furthermore, those skilled in the art may combine different embodiments or examples and features of different embodiments or examples described in this specification, unless they are contradictory to each other.

Although embodiments of the present disclosure are illustrated and described above, it can be understood that the above embodiments are illustrative and should not be construed as limitations of the present disclosure. Those skilled in the art can make changes, modifications, substitutions, and variations to the above embodiments within the scope of the present disclosure.

What is claimed is:

1. A culture medium composition for inducing differentiation of pluripotent stem cells into CD34⁺ hematopoietic stem/progenitor cells, the culture medium composition comprising:
a first differentiation medium added in a first differentiation stage;
a second differentiation medium added in a second differentiation stage; and
a third differentiation medium added in a third differentiation stage, wherein:
the first differentiation medium comprises a first basal medium and BMP4 and a GSK-30 inhibitor;
the second differentiation medium comprises a second basal medium and optionally UM171, and does not comprise IL-3; and
the third differentiation medium comprises a third basal medium, VEGF, bFGF, SCF, FLT3L, and TPO, and does not comprise IL-3.

2. The culture medium composition according to claim 1, wherein:
the pluripotent stem cells are iPSCs or commercially available embryonic stem cells;
optionally, the first basal medium comprises at least one selected from mTeSR™1, TeSR™2, TeSR™-AOF, Essential 8™ Culture Media, NutriStem® hESC XF, or StemFit® Feeder-Free Stem Cell Culture Media;
optionally, the first basal medium is mTeSR™1 or TeSR™-AOF medium;
optionally, the second basal medium is StemPro™-34 SFM complete medium;
optionally, the third basal medium is StemPro™-34 SFM complete medium.

3. The culture medium composition according to claim 1, wherein:
the first differentiation medium further comprises a Rock inhibitor,
optionally, the Rock inhibitor comprises at least one selected from Y27632 or HB-100.

4. The culture medium composition according to claim 1, wherein:
the second differentiation medium further comprises VEGF, bFGF, and BMP4;
optionally, the second differentiation medium further comprises an induction enhancer;
optionally, the induction enhancer comprises at least one selected from a GSK-3β inhibitor or SR1;
optionally, the GSK-3β inhibitor comprises at least one selected from CHIR99021, NP031112, AT7519, TWS119, SB216763, CHIR-98014, AZD1080, SB415286, LY2090314, (E/Z)-GSK-3β inhibitor 1, KY19382, Alsterpaullone, BIO-acetoxime, IM-12, 1-Azakenpaullone, or Indirubin;
optionally, the second differentiation medium further comprises ITS-X, β-mercaptoethanol, ascorbic acid, and GlutaMAX.

5. The culture medium composition according to claim 1, wherein the third differentiation medium further comprises ITS-X, β-mercaptoethanol, ascorbic acid, and GlutaMAX.

6. The culture medium composition according to claim 1, wherein
BMP4 is used at a concentration ranging from 5 ng/mL to 100 ng/mL;
optionally, the GSK-3β inhibitor is CHIR99021, and CHIR99021 is used at a concentration ranging from 1 M to 20 M;
optionally, the Rock inhibitor is Y27632, and Y27632 is used at a concentration ranging from 1 μM to 20 μM;
optionally, VEGF is used at a concentration ranging from 5 ng/mL to 100 ng/mL;
optionally, bFGF is used at a concentration ranging from 5 ng/mL to 100 ng/mL;
optionally, SCF is used at a concentration ranging from 5 ng/mL to 100 ng/mL;
optionally, TPO is used at a concentration ranging from 1 ng/mL to 100 ng/mL;
optionally, FLT3L is used at a concentration ranging from 1 ng/mL to 200 ng/mL, preferably from 1 ng/mL to 50 ng/mL;
optionally, UM171 is used at a concentration ranging from 0 nM to 1 μM;
optionally, the induction enhancer is SR1, and SR1 is used at a concentration ranging from 0 μM to 2 μM.

7. A culture medium composition for inducing differentiation of pluripotent stem cells into NK cells, the culture medium composition comprising:
the first differentiation medium, the second differentiation medium, and the third differentiation medium in the culture medium composition according to claim 1; and
a fourth differentiation medium, wherein:
the fourth differentiation medium comprises a fourth basal medium, IL-7, IL-15, FLT3L, SCF, and optionally IL-3; and
the second differentiation medium comprises UM171,
optionally, the second differentiation medium or the third differentiation medium further comprises an SPHK2 inhibitor.

8. The culture medium composition according to claim 7, wherein:
the pluripotent stem cells are iPSCs or commercially available embryonic stem cells;
optionally, the fourth basal medium is DMEM/F12;
optionally, UM171 is used at a concentration ranging from 10 nM to 1 μM;
optionally, the SPHK2 inhibitor is ABC294640;
optionally, ABC294640 is used at a concentration ranging from 1 M to 50 M;
optionally, IL-3 is used at a concentration ranging from 0 ng/mL to 10 ng/mL;
optionally, IL-7 is used at a concentration ranging from 0.1 ng/mL to 30 ng/mL;
optionally, IL-15 is used at a concentration ranging from 1 ng/mL to 10 ng/mL;
optionally, FLT3L is used at a concentration ranging from 1 ng/mL to 200 ng/mL, preferably from 1 ng/mL to 50 ng/mL;
optionally, SCF is used at a concentration ranging from 5 ng/mL to 100 ng/mL;
optionally, the fourth differentiation medium further comprises human serum albumin and at least one of serum substitute, human AB serum, or FBS.

9. The culture medium composition according to claim 7, wherein:
the fourth differentiation medium further comprises at least one of NAD⁺, HLA-C, SB203580, or IL-2;
optionally, NAD⁺ is used at a concentration ranging from 1 μM to 500 μM;
optionally, HLA-C is used at a concentration ranging from 0.05 ng/mL to 1 ng/mL;
optionally, SB203580 is used at a concentration ranging from 1 M to 50 M;
optionally, IL-2 is used at a concentration ranging from 100 IU to 5000 IU.

10. A method for preparing CD34⁺ hematopoietic stem/progenitor cells, the method comprising:
A. obtaining pluripotent stem cells in a single-cell state;
B. culturing the pluripotent stem cells in the single-cell state under a first culture condition, to obtain a first cell spheroid;
C. culturing the first cell spheroid under a second culture condition, to obtain a second cell spheroid; and
D. culturing the second cell spheroid under a third culture condition, to obtain the CD34⁺ hematopoietic stem/progenitor cells, wherein:
the first culture condition comprises inducing differentiation of the pluripotent stem cells using a first differentiation medium,
the second culture condition comprises inducing differentiation of the first cell spheroid using a second differentiation medium,
the third culture condition comprises inducing differentiation of the second cell spheroid using a third differentiation medium,
the first differentiation medium, the second differentiation medium, and the third differentiation medium are the first differentiation medium, the second differentiation medium, and the third differentiation medium in the culture medium composition for inducing differentiation of pluripotent stem cells into CD34⁺ hematopoietic stem/progenitor cells according to claim 1; and
the pluripotent stem cells are iPSCs or commercially available embryonic stem cells.

11. A method for preparing NK cells, the method comprising:
a. obtaining CD34⁺ hematopoietic stem/progenitor cells using the method according to claim 10;
b. culturing the CD34⁺ hematopoietic stem/progenitor cells under a fourth culture condition, to obtain the NK cells, wherein:
the fourth culture condition comprises inducing differentiation of the CD34⁺ hematopoietic stem/progenitor cells using a fourth differentiation medium,
the fourth differentiation medium comprises a fourth basal medium, IL-7, IL-15, FLT3L, SCF, and optionally IL-3, and
the second differentiation medium comprises UM171,
optionally, the second differentiation medium or the third differentiation medium further comprises an SPHK2 inhibitor.

12. The method according to claim 11, wherein:
UM171 is used at a concentration ranging from 1 nM to 1 μM, and optionally, UM171 is used at a concentration of 35 nM; and
optionally, the SPHK2 inhibitor is ABC294640, and optionally, ABC294640 is used at a concentration ranging from 1 μM to 50 μM.

13. A method for preparing CAR-NK cell, the method comprising:
modifying or genetically editing pluripotent stem cells, to obtain CAR-modified pluripotent stem cells, and
inducing differentiating of the CAR-modified pluripotent stem cells using the method for preparing NK cells according to claim 11, to obtain the CAR-NK cell.

14. The method according to claim 13, wherein the pluripotent stem cells are iPSCs or commercially available embryonic stem cells.


SEQUENCE LISTING (15 sequences)

SEQ ID NO: 1 - DNA, 20 bp, synthetic construct
  source 1..20: mol_type = other DNA
agtttgtgccagggtttttg

SEQ ID NO: 2 - DNA, 20 bp, synthetic construct
  source 1..20: mol_type = other DNA
acttcaccttccctccaacc

SEQ ID NO: 3 - DNA, 22 bp, synthetic construct
  source 1..22: mol_type = other DNA
gtgatcggaaatgacactggag

SEQ ID NO: 4 - DNA, 23 bp, synthetic construct
  source 1..23: mol_type = other DNA
catgttggtcactaacagaagca

SEQ ID NO: 5 - DNA, 20 bp, synthetic construct
  source 1..20: mol_type = other DNA
ccaaagttgtgcggcgtatc

SEQ ID NO: 6 - DNA, 21 bp, synthetic construct
  source 1..21: mol_type = other DNA
caggcggaggatctcattctt

SEQ ID NO: 7 - DNA, 23 bp, synthetic construct
  source 1..23: mol_type = other DNA
ctacaacacctagtacccttgga

SEQ ID NO: 8 - DNA, 22 bp, synthetic construct
  source 1..22: mol_type = other DNA
ggtgaacactgtgctgattaca

SEQ ID NO: 9 - DNA, 20 bp, synthetic construct
  source 1..20: mol_type = other DNA
ggcgtcagagtgggaaatcc

SEQ ID NO: 10 - DNA, 21 bp, synthetic construct
  source 1..21: mol_type = other DNA
ggcaggcagttcacatctacc

SEQ ID NO: 11 - DNA, 23 bp, synthetic construct
  source 1..23: mol_type = other DNA
tatgagcctcgaatccacatagt

SEQ ID NO: 12 - DNA, 22 bp, synthetic construct
  source 1..22: mol_type = other DNA
cctcgttctgataagcagtcac

SEQ ID NO: 13 - DNA, 21 bp, synthetic construct
  source 1..21: mol_type = other DNA
ggagcgagatccctccaaaat

SEQ ID NO: 14 - DNA, 23 bp, synthetic construct
  source 1..23: mol_type = other DNA
ggctgttgtcatacttctcatgg

SEQ ID NO: 15 - protein, 563 aa, synthetic construct
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSLINTTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR